(19) United States
(12) Patent Application Publication
(10) Pub. No.: US 2020/0024315 A1
(43) Pub. Date: Jan. 23, 2020

(54) ANTIMICROBIAL PEPTIDES AND METHODS OF TREATING GRAM-NEGATIVE PATHOGENS

(71) Applicant: THE REGENTS OF THE UNIVERSITY OF COLORADO, a body corporate, Denver, CO (US)

(72) Inventors: Robert S. HODGES, Aurora, CO (US); Ziqing JIANG, Denver, CO (US)

(21) Appl. No.: 16/495,793

(22) PCT Filed: Mar. 20, 2018
(86) PCT No.: PCT/US2018/023323

§ 371 Date: Sep. 19, 2019

Related U.S. Application Data
(60) Provisional application No. 62/473,813, filed on Mar. 20, 2017.

(51) Int. Cl. C07K 14/47; C07K 14/46; A61K 9/00

(52) U.S. Cl. CPC C07K 14/4723; C07K 14/461; A61K 38/00; A61K 9/0014; C07K 14/463

(57) ABSTRACT

Antimicrobial agents, including antimicrobial peptides (AMPs) and uses thereof. Compositions and methods of using dermaseptin-type and piscidin-type antimicrobial peptide variants that demonstrate activity and improved therapeutic indices against microbial pathogens. The peptide compositions demonstrate the ability to not only maintain or improve antimicrobial activity against bacterial pathogens including Gram-negative microorganisms Acinetobacter baumannii and Pseudomonas aeruginosa, but also significantly decrease hemolytic activity against human red blood cells. Specificity determinants within the AMPs change selectivity from broad spectrum antimicrobial activity to Gram-negative selectivity.

### CROSS-REFERENCE TO RELATED APPLICATIONS

This application claims the benefit of U.S. Provisional Patent Application Ser. No. 62/473,813, filed Mar. 20, 2017, the entire disclosure of which is hereby incorporated herein by reference.

### FIELD OF THE INVENTION

This disclosure relates to the field of antimicrobial peptides (AMPS) and treatments for microbial infections.

### BACKGROUND

The explosion of bacterial resistance to traditional antibiotics and a rapid increase in the incidence of multi-drug resistant microbes have created an urgency to develop new classes of antimicrobial agents. There are now “Superbugs” resistant to most or all antibiotics (Coast, J., et al. Health Economics 1996, 5:217-26). The Infectious Diseases Society of America has reported that two-thirds of all health care associated infections are caused by six multi-drug resistant organisms referred to as “ESKAPE” pathogens consisting of two Gram-positive organisms, Enterococcus faecium and Staphylococcus aureus, and four Gram-negative organisms, Klebsiella pneumonia, Acinetobacter baumannii, Pseudomonas aeruginosa and Enterbacter species (Science Daily 9 Dec. 2008, sciencedaily.com/releases/2008/12/081201105706.htm). A recent study in Mexico (Garza-Gonzalez, E., et al. Chemotherapy 2010, 56:275-79) demonstrated dramatic increases in the incidence of antibiotic-resistant species. Of 550 clinical isolates of A. baumannii and 250 clinical isolates of P. aeruginosa, 74% of A. baumannii, and 34% of P. aeruginosa were multi-drug resistant.

Polymyxin B and Polymyxin E (Colistin) are cationic peptides consisting of a cyclic heptapeptide with a tripeptide side chain acylated by a fatty acid chain at the amino terminus. These antibiotics were heavily used in the 1960s, but in the 1970s their clinical use was limited due to serious issues of nephrotoxicity and neurotoxicity (Biswas, S., et al. Expert Rev. Anti. Infect. Ther. 2012, 10:917-34; Yu, Z. et al, BioMed Res. Intl. 2015, dx.doi.org/10.1155/2015/679109). The revival of these two peptides began in the mid-1990s, due to the lack of novel antibiotics effective against the increasingly-prevalent multi-drug resistant Gram-negative bacteria. Thus, these compounds have become antibiotics of last resort, needed for drug resistant bacteria but associated with a high incidence of toxicity. Resistance to these polymyxins could become a major global health challenge because virtually no new antibiotics are currently available for treating serious Gram-negative infections caused by polymyxin-resistant “superbugs.” Accordingly, there is a great need for additional therapeutic antimicrobial treatments effective against drug-resistant organisms.

### SUMMARY

Antimicrobial peptides (AMPs) are produced by bacteria, fungi, plants, insects, amphibians, crustaceans, fish and mammals, including humans, either constitutively or in response to the presence of a microbe (Jenssen, H., et al. Clin Microbiol Rev. 2006, 19, 491-511). AMPs are rapidly bactericidal and generally have broad-spectrum activity. It is believed that the antimicrobial mechanism of action of cationic AMPs does not involve a stereoselective interaction with a chiral enzyme or lipid or protein since enantiomeric forms of AMPs with all-D-amino acids have shown equal activities compared to their all-L-enantiomers (Chen, Y., et al. Chem. Biol. Drug Des. 2006, 67:162-73). Because their mode of action apparently involves non-specific interactions with the cytoplasmic membrane of bacteria, bacteria rarely develop resistance to them. Additionally, all D-enantiomer peptides are resistant to proteolytic enzyme degradation, which enhances their potential use as therapeutic agents in mammals. Unfortunately, native AMPs lack specificity between prokaryotic and eukaryotic cells, and are therefore too toxic to be used for systemic treatment of bacterial infections. This toxicity, which manifests as drug- and dose-limiting hemolysis of human red blood cells, has limited the development of a new class of antimicrobial agents based on these AMPs.

To overcome the toxicity problem, the present inventors developed the design concept of “specificity determinants,” which refers to substituting positively charged residues in the center of the non-polar face of the amphipathic α-helical AMPs to create selectivity between eukaryotic and prokaryotic membranes. The objective of substituting “specificity determinants” is to maintain or enhance antimicrobial activity while decreasing or eliminating hemolytic activity or cell toxicity to mammalian cells. Toxicity, the ability to lyse mammalian cells, is most frequently expressed as hemolytic activity against human red blood cells. The present inventors have previously used an antimicrobial peptide in the D-enantiomeric configuration with one lysine substitution (“D1 (K13)”) as a starting point to design antimicrobial peptides with enhanced biologic properties for Gram-negative pathogens only, rather than broad-spectrum activity (Jiang, Z., et al., Chem. Biol. Drug Des. 2011, 77:225-40). The number and location of positively charged residues on the polar and non-polar face of this AMP were studied, ultimately resulting in the development of four new antimicrobial peptides with improvements in antimicrobial activity against Gram-negative pathogens and dramatic reductions in hemolytic activity and therefore unprecedented improvements in therapeutic indices.

The inventors have also studied the antimicrobial peptides piscidin 1 and dermaseptin S4 for substitution of one or two amino acid(s) to lysine(s) at different positions in the center of their nonpolar faces to investigate the generality of the “specificity determinant” design concept to enhance or maintain antimicrobial activity and significantly improve the therapeutic index (Jiang, Z., et al., Pharmaceuticals 2014, 7:366-391).

The inventors also prepared variants in two native AMPs piscidin 1 (isolated from mast cells of hybrid striped bass—Morone saxatilis male×Morone chrysops female) and dermaseptin S4 (isolated from the skin of tree-dwelling, South American frogs of the Phyllomedusa species) (Jiang, Z., et al., Proceedings of the 24^{th} American Peptide Symposium. In Enabling Peptide Research from Basic Research to Drug Discovery, Orlando, Fla. (V. Srivastava, A. Yudin and M. Lebl, editors) 2015, pp. 245-48). These variant peptides were tested for their antimicrobial activity against two different pathogens: 11 and 20 diverse clinical isolates of A. baumannii, and Staphylococcus aureus (12 Methicillin-sensitive S. aureus strains and 8 Methicillin/Oxacillin-resistant S. aureus strains), respectively. These studies showed that substitution of “specificity determinant(s)” in broad spectrum AMPs, encode selectivity for Gram-negative pathogens and simultaneously remove both Gram-positive activity and hemolytic activity of these two, diverse amphipathic alpha-helical AMPs which differ dramatically in amino acid composition, net positive charge and amphipathicity.

This disclosure provides highly effective and specific antimicrobial agents comprising peptides and peptide-containing compositions, and methods of inhibiting microorganisms, and treating a subject in need of antimicrobial therapy.

The antimicrobial peptides (AMPs) and compositions of this disclosure demonstrate activity and improved therapeutic indices against bacterial pathogens. These AMPs demonstrate the ability to not only maintain or improve antimicrobial activity against Gram-negative bacterial pathogens, but also significantly decrease the hemolysis of human red blood cells. Thus, improved therapeutic indices are achieved by AMPs of this disclosure.

To overcome the significant mammalian toxicity of most of the known AMPs, the inventors developed the design concept of the “specificity determinant,” which refers to the substitution of positively charged amino acid residue(s) in the non-polar face of amphipathic alpha-helical or cyclic beta-sheet antimicrobial peptides to create selectivity between eukaryotic and prokaryotic membranes; that is, antimicrobial activity is maintained and hemolytic activity or cell toxicity to mammalian cells is substantially decreased or eliminated.

The inventors selected piscidin 1 and dermaseptin S4 as examples of native AMPs to substitute positively charged amino acid(s) at different positions in their non-polar faces to enhance or maintain antimicrobial activity and significantly improve the therapeutic index.

This disclosure provides peptide antimicrobial agents and antimicrobial peptide compositions, as well as methods of inhibiting microorganisms and treating microbial infections, particularly infections by drug-resistant microorganisms. A subject may be treated by administering an AMP or a composition comprising an AMP of this disclosure. The antimicrobial peptides (AMPs) of this disclosure demonstrate activity and improved therapeutic indices against bacterial pathogens. These AMPs may demonstrate the ability to not only maintain or improve antimicrobial activity against bacterial pathogens, including Gram-negative microorganisms such as Acinetobacter baumannii and Pseudomonas aeruginosa, but also significantly decrease hemolytic activity against human red blood cells. Thus, the AMPs of this disclosure display improved therapeutic indices.

Isolated antimicrobial peptides (AMPs) of this disclosure comprise the amino acid sequence (referring to the single-letter amino acid code) of:

(SEQ ID NO: 1)
X¹-L-X²-X³-L-L-X⁴-X⁵-L-X⁶-X⁷-A-X⁸-X⁹-X¹⁰-X¹¹-L-X¹²-X¹³-
L-L-X¹⁴-A-L-X¹⁵-X¹⁶

Wherein:

each residue is in the D-enantiomeric form;

X¹ is an amino acid in the D-enantiomeric form selected from Lysine (K; Lys), Arginine (R; Arg), Ornithine (O; Orn), and Alanine (A; Ala);

each of X², X³, X⁴, X⁶, X⁷, X¹², X¹⁴, and X¹⁵ are independently, amino acids in the D-enantiomeric form selected from Lysine (K; Lys), Arginine (R; Arg), Ornithine (O; Orn), Diaminobutyric acid (Dbu), and Serine (S; Ser);

each of X⁵, X¹³, X¹⁶ are independently, amino acids in the D-enantiomeric form selected from Lysine (K; Lys), Arginine (R; Arg), Ornithine (O; Orn), Diaminobutyric acid (Dbu), and Serine (S; Ser), and Threonine (T; Thr);

each of X⁸, X¹¹ are independently, amino acids in the D-enantiomeric form selected from Lysine (K; Lys), Serine (S; Ser), and Threonine (T; Thr), Arginine (R; Arg), Ornithine (O; Orn), Alanine (A; Ala), Diaminobutyric acid (Dbu), and Diaminopropionic acid (Dpr); and,

each of X⁹ and X¹⁰ are independently, amino acids in the D-enantiomeric form selected from Lysine (K; Lys), Arginine (R; Arg), Ornithine (O; Orn), Serine (S; Ser), and Alanine (A; Ala);

These AMPs may comprise two positively charged residues on the non-polar face (i.e., “specificity determinants”) and 5 positively charged residues on the polar face. Thus, the total charge on these AMP molecules of this disclosure is +7.

The peptides of this disclosure include residues that disrupt the continuous hydrophobic surface that stabilizes the alpha-helical structure of AMPs that lack the “specificity determinants” (such as the naturally occurring peptides Piscidin 1 and/or Dermaseptin S4, and/or the all D-enantiomeric forms of these naturally occurring peptides).

In one embodiment, the peptides of this disclosure have dramatically reduced α-helical structure in aqueous environment but inducible α-helical structure when the peptides are in a hydrophobic environment to mimic the hydrophobicity of membrane.

In another embodiment, the peptides of this disclosure may include residues that reduce the hydrophobicity on the non-polar face and overall hydrophobicity of the peptide molecule (as measured by retention time at 25° C. by reversed-phase chromatography (RP-HPLC).

In another embodiment, the peptides of this disclosure may include residues that control overall hydrophobicity of the peptide as measured by relative retention time in RP-HPLC through the temperature range 5° C. to 77° C. These parameters are compared to the biological activity of the peptides.

In another embodiment, the peptides of this disclosure may include residues that dramatically reduce peptide self-association in aqueous conditions (as measured by the temperature profiling in RP-HPLC procedure from 5° C. to 77° C. described in the Examples section of this disclosure, particularly Example 4).

In another embodiment, the peptides of this disclosure may have dramatically reduced toxicity to normal cells (as measured by hemolytic activity to human red blood cells at 37° C. after 18 hours). In this context, HC₅₀ hemolytic activity is defined as the concentration of peptide (μM) that results in 50% hemolysis after 18 hours at 37° C. A skilled person will appreciate that it is advantageous that this value is as large as possible. In particular, hemolytic activity may be measured by the procedure described in the Examples section of this disclosure, particularly Example 6. If 50% hemolysis cannot be achieved, HC₃₀ values (30% hemolysis) are specifically quoted. In a further embodiment, the peptides of this disclosure exhibit hemolytic activity expressed as HC₅₀ value of greater than 100 μg/ml, in particularly 300 μg/ml or greater, more particularly greater than 5000 μg/ml. In a yet further embodiment, the peptides of this disclosure exhibit less than 10% hemolysis at 500 μg/ml.

In another embodiment, the peptides of this disclosure may have similar or substantially enhanced antimicrobial activity (compared to AMPs lacking specificity determinants, such as the naturally occurring peptides Piscidin 1 and/or Dermaseptin S4, and/or the all D-enantiomeric forms of these naturally occurring peptides), and particularly with respect to bactericidal activity towards Gram-negative microbes. Antimicrobial activity is expressed as the MIC (the minimum concentration of peptide required to inhibit growth of bacteria after 24 hour at 37° C.). In particular, antimicrobial activity may be measured by the procedure described in the Examples section of this disclosure, particularly Example 5. In a further embodiment, the peptides of this disclosure exhibit antimicrobial activity expressed as the geometric mean MIC value (MIC_{GM}) of less than 6 μg/ml or less than 2 μM. In a particular embodiment, the peptides of this disclosure exhibit antimicrobial activity expressed as the geometric mean MIC (MIC_{GM}) of about 2 μg/ml or about 0.6 μM.

In another embodiment of the invention, the peptides of this disclosure may have dramatically improved therapeutic indices (calculated by the ratio of hemolytic activity and antimicrobial activity (MIC)) compared to AMPS lacking specificity determinants, such as the naturally occurring peptides Piscidin 1 and/or Dermaseptin S4, and/or the all D-enantiomeric forms of these naturally-occurring peptides. The ratio of hemolytic activity/antimicrobial activity defines the therapeutic index for a given AMP and is a measure of specificity of the AMP for bacterial membranes. A skilled person will appreciate that typically the higher the therapeutic index, the more specific the AMP is for prokaryotic cells. In a further embodiment, the peptides of this disclosure exhibit therapeutic index values (expressed as HC₅₀/MIC_{GM}) greater than 200, in particular greater than 2000. A skilled person will appreciate that the therapeutic index is a ratio and the units of measurement of HC₅₀ and MIC_{GM} are required to be consistent.

In another embodiment, the peptides of this disclosure may have antimicrobial selectivity for Gram-negative pathogens resulting from significantly decreased Gram-positive activity and hemolytic activity (compared to AMPs lacking specificity determinants, such as the naturally occurring peptides Piscidin 1 and/or Dermaseptin S4, and/or the all D-enantiomeric forms of these naturally occurring peptides). The peptides of this disclosure may have antimicrobial activity against A. baumannii bacterial strains resistant to polymyxin B and/or polymyxin E (Colistin) antibiotics. The peptides of this disclosure may discriminate between eukaryotic and prokaryotic cell membranes. The peptides of this disclosure may have antimicrobial activity even in the presence of human serum.

In particular, the following peptides of this disclosure were designed and tested:
A. SEQ ID NO:3 [D26 (with two specificity determinants, Lys 13 and Lys 16)] and SEQ ID NO:2 [D26 (K13A, K16A) (without two specificity determinants)], and 5 analogs of SEQ ID NO:3 (D26) denoted SEQ ID NO:4 (D51), SEQ ID NO:5 (D52), SEQ ID NO:6 (D53), SEQ ID NO:7 (D54) and SEQ ID NO:8 (D55) where the location of the 5 Lys residues on the polar face were varied (Table 1A, FIG. 1);
B. four analogs with variation of the type of positively charged residue on the non-polar face of SEQ ID NO:5 [D52 (Lys 13, Lys 16)]; SEQ ID NO:11 [D56 (Orn 13, Orn 16)], SEQ ID NO:12 [D57 (Dbu 13, Dbu 16)] and SEQ ID NO:13 [D58 (Arg 13, Arg 16)] (Table 1B, FIG. 2);
C. four analogs with variation the type of positively charged residue on the polar face of SEQ ID NO:5 [D52 (Lys 3, 4, 22, 25 and 26)]; SEQ ID NO:15 [D59 (Orn 3, 4, 22, 25 and 26)]; SEQ ID NO:16 [D60 (Dbu 3, 4, 22, 25 and 26)] and SEQ ID NO:17 [D61 (Arg, 3, 4, 22, 25, 26)] (Table 1C, FIG. 2).

The peptides listed above were all characterized by circular dichroism spectroscopy, temperature profiling in RP-HPLC, relative hydrophobicity differences by RP-HPLC, hemolytic activity and antimicrobial activity to select the best analogs in the three categories, location of Lys residues on the polar face; type of positively charged residue on the non-polar face as specificity determinants and the type of positively charged residue to use on the polar face.

The peptides listed above were screened against seven strains of Acinetobacter baumannii resistant to colistin and polymyxin B.

In aspects of the claimed antimicrobial peptides, the AMPS of this disclosure comprise the peptides of the following tables (each of which comprises the listed amino acids, set forth in the one-letter amino acid code, all in the D-enantiomeric form):

TABLE 1A
Peptides used in this study

| | | | SEQ |
|---|---|---|---|
| | | Sequence^{b} | ID |
| Laboratory Peptide | Length | 1 3 4 7 8 1011 131415 16 18 19 22 25 26 | NO |
| Name^{a} | (mer) | X¹LX²X³LLX⁴X⁵LX⁶X⁷AX⁸X⁹X¹⁰X¹¹LX¹²X¹³LLX¹⁴ALX¹⁵X¹⁶ | 1 |

Without specificity determinants

| | | | |
|---|---|---|---|
| D26 (K13A, K16A) | 26 | Ac-KLKSLLSTLSSAAKKALSTLLSALSK-amide | 2 |

With specificity determinants

| | | | |
|---|---|---|---|
| D26 | 26 | Ac-KLKSLLSTLSSAKKKKLSTLLSALSK-amide | 3 |
| D51 | 26 | Ac-ALKKLLSTLSSAKKKKLSTLLSALSK-amide | 4 |
| (D26 (K1A/S4K)) | | | |
| D52 | 26 | Ac-ALKKLLSTLSSAKSSKLSTLLKALKK-amide | 5 |
| (D26 (K1A/S4K/K14S/ | | | |
| K15S/S22K/S25K)) | | | |
| D53 | 26 | Ac-ALSSLLKKLSSAKSSKLSTLLKALKK-amide | 6 |
| (D26 (K1A/K3S/S7K/ | | | |
| T8K/K14S/K15S/ | | | |
| S22K/S25K)) | | | |
| D54 | 26 | Ac-ALSSLLSTLKKAKSSKLSTLLKALKK-amide | 7 |
| (D26 (K1A/K3S/S10K/ | | | |
| S11K/K14S/K15S/ | | | |
| S22K/S25K)) | | | |
| D55 | 26 | Ac-ALSSLLSTLKKAKSSKLKKLLKALSS-amide | 8 |
| (D26 (K1A/K35/S10K/ | | | |
| S11K/K14S/K15S/ | | | |
| Sl8K/T19K/S22K/K26S) | | | |
| D16 | 26 | Ac-KLKSLLKTLSKAKKKKLKTLLKALSK-amide | 9 |

^{a}The ′D′ denotes that all amino acid residues in each peptide are in the D-conformation.

The series of peptides shown in Table 1B were designed and tested to show the effects of substitutions to the specificity determinants at positions 13 and 16 of these 26-mer AMPS. The lysine residues at these positions were substituted with Ornithine (Orn), Diaminobutyric acid (Dbu), or Arginine (Arg).

TABLE 1B
D52 non-polar face substitutions

| | Sequence^{b} | |
|---|---|---|
| Laboratory Peptide | 13 16 | SEQ ID NO |
| Name | ALKKLLSTLSSA X⁸ SS X¹¹ LSTLLKALKK | 10 |
| D52 | Ac-ALKKLLSTLSSA(Lys)SS(Lys)LSTLLKALKK-amide | 5 |
| D56 | Ac-ALKKLLSTLSSA(Orn)SS(Orn)LSTLLKALKK-amide | 11 |
| (D52 (K13Orn/K16Orn)) | | |
| D57 | Ac-ALKKLLSTLSSA(Dbu)SS(Dbu)LSTLLKALKK-amide | 12 |
| (D52 (K13Dbu/K16Dbu)) | | |
| D58 | Ac-ALKKLLSTLSSA(Arg)SS(Arg)LSTLLKALKK-amide | 13 |
| (D52 (K13Arg/K16Arg)) | | |

b

Thus, the isolated antimicrobial peptides (AMPS) of this disclosure may include peptides having the the amino acid sequence (referring to the single-letter amino acid code), entirely in the D-enantiomeric form:

(SEQ ID NO: 10)
ALKKLLSTLSSA X⁸ SS X¹¹ LSTLLKALKK

wherein each of X⁸, X¹¹ are independently amino acids in the D-enantiomeric form selected from Lysine (K; Lys), Arginine (R; Arg), Ornithine (O; Orn), and Diaminobutyric acid (Dbu).

The series of peptides shown in Table 1C were designed and tested to show the effects of substitutions to lysine residues on the polar face (at positions 3, 4, 22, 25, and 26) of these 26-mer AMPS. The lysine residues at these positions were substituted with Ornithine (Orn), Diaminobutyric acid (Dbu), or Arginine (Arg). This analysis was conducted by substituting the polar face lysine residues of peptide D52 with Ornithine (Orn), Diaminobutyric acid (Dbu), or Arginine (Arg), but this can also be done by substituting the polar face lysine residues of any one of peptides D51, D53, D54, and D55.

TABLE 1C
D52 polar face substitutions

| | Sequence^{c} | |
|---|---|---|
| | 3 4 22 25 26 | SEQ ID NO |
| Peptide Name | AL X² X³ LLSTLSSAKSSKLSTLLX¹⁴ AL X¹⁵ X¹⁶ | 14 |
| D52 | Ac-ALLysLysLLSTLSSAKSSKLSTLLLysALLysLys-amide | 5 |
| D59 | Ac-ALOrnOrnLLSTLSSAKSSKLSTLLOrnALOrnOrn-amide | 15 |
| (D52 (K3Orn/K4Orn/ | | |
| K22Orn/ | | |
| K25Orn/K26Orn)) | | |
| D60 | Ac-ALDbuDbuLLSTLSSAKSSKLSTLLDbuALDbuDbu-amide | 16 |
| (D52 (K3Dbu/K4Dbu/ | | |
| K22Dbu/ | | |
| K25Dbu/K26Dbu)) | | |
| D61 | Ac-ALArgArgLLSTLSSAKSSKLSTLLArgALArgArg-amide | 17 |
| (D52 (K3Arg/K4Arg/ | | |
| K22Arg/ | | |
| K25Arg/K26Arg)) | | |

^{c}Peptide sequences are shown using the one-letter code (or three-letter code for Orn, Dbu and Arg); Ac denotes N^{α}-acetyl and -amide denotes C^{α}-amide. Specificity determinants (K) at positions 13 and 16 are held constant in the center of the non-polar face. Lysine residues on the polar face, at positions 3,4, 22, 25 and 26, are Lys residues for peptide D52; Orn residues for peptide D59; Dbu residues for peptide D60; and Arg residues for peptide D61.

Thus, the isolated antimicrobial peptides (AMPs) of this disclosure may include peptides having the the amino acid sequence (referring to the single-letter amino acid code), entirely in the D-enantiomeric form:

(SEQ ID NO: 14)
AL X² X³ LLSTLSSAKSSKLSTLLX¹⁴ AL X¹⁵ X¹⁶

wherein each of X², X³, X¹⁴, X¹⁵, X¹⁶ are independently amino acids in the D-enantiomeric form selected from Lysine (K; Lys), Arginine (R; Arg), Ornithine (O; Orn), and Diaminobutyric acid (Dbu).

Another aspect of this disclosure provides pharmaceutical compositions comprising at least one of the antimicrobial peptides of this disclosure, and a pharmaceutically acceptable carrier. In aspects of the claimed pharmaceutical compositions the compositions may include one or more AMPs of SEQ ID NOs: 1, 3-8 and 10-17.

Another aspect provides a method of preventing or treating an infection in a subject, including administering a therapeutically effective amount of a composition to the subject, wherein the composition comprises at least one antimicrobial peptide of this disclosure, and a pharmaceutically acceptable carrier. In aspects of the claimed methods the infecting microorganism is a Gram-negative bacteria.

In these methods, the infecting microorganism may be an antibiotic resistant microbe. The antibiotic resistant microbe may be a Gram-negative, antibiotic-resistant Acinetobacter baumannii or Pseudomonas aeruginosa pathogen. Alternatively or additionally, the antibiotic infecting microorganism may be a drug-resistant (such as a polymyxin B and/or polymyxin E (Colistin)-resistant) Gram-negative pathogen, or a polymyxin B and/or polymyxin E sensitive Gram-negative pathogen.

This disclosure also provides methods of inhibiting a microorganism, comprising contacting the microorganism with a composition comprising at least one AMP of this disclosure. In these methods, the AMP may be one or more of the peptides having an amino acid sequence of any one of SEQ ID NOs:1, 3-8 and 10-17.

One aspect of this disclosure provides an antimicrobial peptide (AMP) comprising an amino acid sequence having at least 85%, or at least 90% or at least 95% homology with a peptide selected from the group consisting of SEQ ID NOs: 1, 3-8 and 10-17, or functional analogues, derivatives or fragments thereof, or pharmaceutically-acceptable salts thereof.

In a preferred aspect of the claimed methods, the amino acid sequence of the administered AMP comprises the sequence of SEQ ID NO:3 In these methods, the AMP inhibits propagation of a prokaryote. The prokaryote may be a Gram-negative bacterium, which may include at least one of A. baumannii and P. aeruginosa bacterium.

In one embodiment, the AMPs of this disclosure may exhibit a therapeutic index (calculated by the ratio of hemolytic activity to antimicrobial activity (MIC)) of at least 70. In a further embodiment, the AMPs of this disclosure may exhibit a therapeutic index of between 70 and 1600.

In another embodiment, the AMPs of this disclosure may exhibit at least a 10-fold increased selectivity for Gram-negative bacteria over Gram-positive bacteria. The AMP may exhibit between a 10-fold and a 90-fold increase in selectivity for Gram-negative bacteria over Gram-positive bacteria. In these selectivity measurements, the Gram-negative bacteria may be A. baumannii and the Gram-positive bacteria may be Staphylococcus aureus. As shown in Table 8 of this disclosure, AMPs of this disclosure may be completely selective for Gram-negative bacteria, and this may include complete selectivity for the Gram-negative bacteria A. baumannii. For example, the AMP of SEQ ID NO:3 (D26) is selective for the Gram-negative pathogen Acinetobacter baumannii and inactive against Pseudomonas aeruginosa, demonstrating that the inventors have designed a Gram-negative selective AMP. In this case, the Gram-negative selective AMP is selective for Acinetobacter baumannii.

In another embodiment, the AMPs of this disclosure having the sequence of any one of SEQ ID NOs: 1, 3-8 and 10-17 may exhibit at least a 30-fold decrease in hemolysis of human red blood cells (measured as HC₃₀—the concentration of peptide that results in 30% hemolysis after 18 h at 37° C.) compared to hemolysis exhibited by Piscidin 1 and/or Dermaseptin S4.

Another aspect of this disclosure provides a pharmaceutical composition comprising at least one AMP of this disclosure and a pharmaceutically acceptable carrier. The pharmaceutical composition may be a mono-phasic pharmaceutical composition suitable for parenteral or oral administration consisting essentially of a therapeutically-effective amount of at least one AMP of this disclosure, and a pharmaceutically acceptable carrier. In these embodiments, the AMP may be one or more of the peptides having the sequence of SEQ ID NOs: 1, 3-8 and 10-17.

Another aspect of this disclosure provides methods of preventing or treating a microbial infection comprising administering to a subject in need thereof of a therapeutically effective amount of at least one AMP of this disclosure, or a pharmaceutical composition comprising the same. In these methods, the AMP administered may be one or more of the peptides having the sequence of SEQ ID NOs: 1, 3-8 and 10-17. In these methods, the microbial infection may be the result of an infecting bacteria, fungi, virus, or protozoa. The microbial infection may be a bacterial infection. The bacterial infection may be a Gram-negative bacterial infection. The bacterial infection may be an antibiotic resistant bacterial infection. The infecting microorganism may be at least one of Pseudomonas aeruginosa, Acinetobacter baumannii. The infecting microorganism may be an antibiotic- or multi drug-resistant Pseudomonas aeruginosa or Acinetobacter baumannii bacteria.

In these methods, the administration of the peptide or pharmaceutical composition may be made by an administration route selected from oral, topical, intravenous, intraperitoneal, intramuscular, intradermal, intrasternal, intraarticular injection, or intrathecal. The peptides or pharmaceutical compositions of this disclosure may be administered in conjunction with one or more additional antimicrobial agents.

This disclosure also provides methods of preventing a microbial infection in an individual at risk of developing an infection comprising administering an effective amount of at least one AMP of this disclosure, or a pharmaceutical composition comprising the same, to an individual in need thereof. The individual may be a surgical patient. The individual may be a hospitalized patient.

This disclosure also provides methods of combating a bacterial infection in a patient comprising applying at least one AMP of this disclosure, or a pharmaceutical composition comprising the same, to a body surface of the patient. The body surface may be a wound. The composition may be applied following an operation or surgery.

This disclosure also provides at least one AMP of this disclosure, or a pharmaceutical composition comprising the same, for use in the treatment of a microbial infection. This disclosure also provides the use of at least one peptide of this disclosure, or a pharmaceutical composition comprising the same, in the manufacture of a medicament for the prevention or treatment of a microbial infection.

### BRIEF DESCRIPTION OF THE FIGURES

FIG. 1 shows helical wheel (upper panels) and helical net (lower panels) representations of our helical AMPs with “specificity determinants” on the non-polar face. In the helical wheels the non-polar face is indicated as a yellow arc (Leu residues are colored yellow and two Lys specificity determinants are colored pink). The polar face is indicated as a black arc (Lys residues are colored blue). In the helical nets, the residues on the polar face are boxed (Lys residues are colored blue) and the residues on the non-polar face are circled (Leu residues are colored yellow and two Lys specificity determinants are colored pink). The location of the five positively charged Lys residues on the polar face is different between peptides D51, D52, D53, D54, and D55. The potential i to i+3 or i to i+4 electrostatic repulsions between positively charged residues are shown on black dotted lines. The i to i+3 or i to i+4 hydrophobic interactions between large hydrophobes are shown as solid black lines.

FIG. 2A and FIG. 2B uses the same helical wheel and net representations to compare the sequences of D26 (with specificity determinants) [SEQ ID NO: 3] and D26 (K13A/K16A) without specificity determinants [SEQ ID NO:2], D52 [SEQ ID NO: 5], which contains two specificity determinants (K13 and K16), with analogs of D52 [SEQ ID NO: 10 and SEQ ID NO: 14]

FIG. 3 depicts a proposed mechanism of temperature profiling by RP-HPLC of amphipathic alpha-helical antimicrobial peptides. Panel A, at low temperatures, peptides capable of self-association in aqueous solution by their non-polar faces establish an equilibrium during RP-HPLC between the bound helical monomer to the hydrophobic stationary phase, the helical monomer in the mobile phase and the helical dimer in the mobile phase during gradient elution. Panel B, at higher temperatures, the population of dimers in the mobile phase during partitioning decreases, increasing the concentration of the monomeric alpha-helical peptide which increases peptide retention time. Panel C, at temperatures beyond the point of maximum retention time the unbound helical peptide in the mobile phase is in equilibrium with the random-coil conformation of the peptide and retention time decreases with further increasing temperature.

FIG. 4 shows circular dichroism (CD) spectroscopy results of D26, D26 control (D26 K13A, K16A) and analogs of D26 (D51-61).

FIGS. 5-8 show temperature profiling (5° C.-77° C.) in RP-HPLC of D26, D26 K13A, K16A and analogs of D26 (D51-D61).

FIG. 9 shows the change in relative hydrophobicity of peptides with substitutions on the polar face varying the location of charged Lys residues compared to D52.

FIG. 10 shows the change in relative hydrophobicity of peptides with substitutions on the polar face varying the type of charged residues compared to D52, where lysine residues in positions 3, 4, 22, 25 and 26 are being substituted with Orn (D59), Dbu (D60) or Arg (D61).

FIG. 11 shows the changed in relative hydrophobicity of peptides with substitutions on the non-polar face compared to D52, where lysine residues in positions 13 and 16 of D52 are being substituted with Orn (D56), Dbu (D57) or Arg (D58).

### DETAILED DESCRIPTION

The terms and phrases used herein have their art-recognized meaning, which can be found by reference to standard texts, journal references and contexts known to those skilled in the art.

As used herein, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “containing” can be used interchangeably. The expression “of any of claims XX-YY” (wherein XX and YY refer to claim numbers) is intended to provide a multiple dependent claim in the alternative form, and in some embodiments, is interchangeable with the expression “as in any one of claims XX-YY.”

Unless defined otherwise, all technical and scientific terms used herein have the same meanings as commonly understood by one of ordinary skill in the pertinent art.

Whenever a range of values is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

The following definitions are provided to clarify use of these terms in the context of this disclosure.

When used herein, the term “amino acid” is intended to refer to any natural or unnatural amino acid, whether made naturally or synthetically, including those in the L- or D-enantiomeric configurations. The term can also encompass amino acid analog compounds used in peptidomimetics or in peptoids. The term can include a modified or unusual amino acid or a synthetic derivative of an amino acid, e.g. diamino butyric acid and diamino propionic acid and the like. The antimicrobial peptides comprise amino acids linked together by peptide bonds. The peptides are in general in alpha helical conformation under hydrophobic conditions. Sequences are conventionally given from the amino terminus to the carboxyl terminus. When all the amino acids are of L-configuration, the peptide is said to be an L-enantiomer. When all the amino acids are of D-configuration, the peptide is said to be a D-enantiomer.

A skilled person will appreciate that when two or more amino acids combine to form a peptide, the elements of water are removed, and what remains of each amino acid is called an amino-acid residue. The amino acid residue is the part of an amino acid that makes it unique from all the others. As such, reference herein to an ‘amino acid’ in the context of an amino acid sequence contained within a peptide will be understood to refer to the respective amino acid residue as appropriate.

Peptides of this disclosure may be substituted, preferably at the N- or C-terminus, by a further moiety. Such moieties may be added to aid the function of the peptide, its targeting or its synthesis, capture or identification, e.g. a label (e.g. biotin) or lipid molecules. The peptides of this disclosure may be chemically modified, for example, post-translationally modified. For example, they may be glycosylated, pegylated or comprise modified amino acid residues. They can be in a variety of forms of polypeptide derivatives, including amides and conjugates with polypeptides. In particular, the amine group at the N-terminus may be substituted with a carboxyl group, such as acetyl, to yield an amide and/or the carboxylic acid group at the C-terminus may be converted to an amide.

Chemically modified peptides also include those having one or more residues chemically derivatized by reaction of a functional side group. Such derivatized side groups include those which have been derivatized to form amine hydrochlorides, amine alkoate salts for example acetate, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives.

A skilled person will appreciate that when both a basic group and an acid group are present in the same molecule, the peptides of the present invention may also form internal salts, e.g., zwitterionic molecules.

The term “hemolytic concentration-50” or HC₅₀ refers to the concentration of peptide (μM) that results in 50% hemolysis of erythrocytes after 18 hours at 37° C.

The term “hemolytic concentration-30” or “HC₃₀” refers to the concentration of peptide (μM) that results in 50% hemolysis of erythrocytes after 18 hours at 37° C.

In particular, hemolytic activity may be measured by the procedure described in the Examples section of this disclosure, particularly Example 6. Hemolytic concentration was determined from a plot of percent lysis versus peptide concentration (microM). For comparison, the inventors also determined the hemolytic activity after 18 hours at 37° C. Hemolysis can be determined with red blood cells (RBC) from various species including human red blood cells (hRBC). A skilled person will appreciate that it is advantageous that the hemolytic concentration value is as large as possible.

The specifically exemplified AMPS of this disclosure are, in some instances, so non-hemolytic to mammalian red blood cells that the HC₅₀ value could not be calculated. Therefore, in these instances the HC₃₀ value was used for testing and comparison purposes to achieve a measure of safety, with respect to hemolysis, that is consistent and comparable across peptides of this disclosure and those known in the art.

The ratio of hemolytic activity/antimicrobial activity defines the therapeutic index for a given AMP and is a measure of specificity of the AMP for bacterial membranes. A skilled person will appreciate that typically the higher the therapeutic index, the more specific the AMP is for prokaryotic cells. The term “therapeutic index” (TI) is the ratio of HC₅₀ over minimal inhibitory concentration (MIC) of an antimicrobial agent. Larger values generally indicate greater antimicrobial specificity.

The term “stability” can refer to an ability to resist degradation, to persist in a given environment, and/or to maintain a particular structure. For example, a peptide property of stability can indicate resistance to proteolytic degradation and to maintain an alpha-helical structural conformation.

The following abbreviations are useful: A, Ala, Alanine; M, Met, Methionine; C, Cys, Cysteine; D, Asp, Aspartic Acid; E, Glu, Glutamic Acid; F, Phe, Phenylalanine; G, Gly, Glycine; H, His, Histidine; I, Ile, Isoleucine; K, Lys, Lysine; L, Leu, Leucine; N, Asn, Asparagine; O, Orn, Ornithine; P, Pro, Proline; Q, Gln, Glutamine; R, Arg, Arginine; S, Ser, Serine; T, Thr, Threonine; V, Val, Valine; W, Trp, Tryptophan; Y, Tyr, Tyrosine; Dbu, 2,4-Diaminobutyric acid; Dpr, 2,3-Diaminopropionic acid; RP-HPLC, reversed-phase high performance liquid chromatography; MIC, minimal inhibitory concentration; MIC_{GM} geometric mean MIC value; HC₃₀ hemolytic concentration-30; HC₅₀ hemolytic concentration-50; CD, circular dichroism spectroscopy; TFE, 2,2,2-trifluoroethanol; TFA, trifluoroacetic acid; RBC, red blood cells; hRBC, human red blood cells.

The term “antimicrobial activity” refers to the ability of a peptide to modify a function or metabolic process of a target microorganism, for example to at least partially affect replication, vegetative growth, toxin production, survival, viability in a quiescent state, or other attribute. The term relates to inhibition of growth of a microorganism. In aspects of the claimed peptides and methods, antimicrobial activity relates to the ability of a peptide to kill at least one bacterial species. The bacterial species may be a Gram-negative bacteria. The term can be manifested as microbicidal or microbistatic inhibition of microbial growth. Antimicrobial activity is expressed as the MIC (the minimum concentration of peptide required to inhibit growth of bacteria after 24 hour at 37° C.). In particular, antimicrobial activity may be measured by the procedure described in the Examples section of this disclosure, particularly Example 5

The phrase “improved biological property” is meant to indicate that a test peptide exhibits less hemolytic activity and/or better antimicrobial activity, or better antimicrobial activity and/or less hemolytic activity, compared to a control peptide (e.g., D-piscidin 1 or D-dermaseptin S4), when tested by the protocols described herein or by any other art-known standard protocols. In general, the improved biological property of the peptide is reflected in the therapeutic index (TI) value which is better than that of the control peptide.

The term “microorganism” herein refers broadly to bacteria, fungi, viruses, and protozoa. In particular, the term is applicable for a microorganism having a cellular or structural component of a lipid bilayer membrane. The membrane may be a cytoplasmic membrane. Pathogenic bacteria, fungi, viruses, and protozoa as known in the art are generally encompassed. Bacteria can include Gram-negative and Gram-positive bacteria in addition to organisms classified in orders of the class Mollicutes and the like, such as species of the Mycoplasma and Acholeplasma genera. Specific examples of Gram-negative bacteria include, but are not limited to, Escherichia coli, Pseudomonas aeruginosa, Acinetobacter baumannii, Salmonella spp., Haemophilus influenzae, Neisseria spp., Vibrio cholerae, Vibrio parahaemolyticus and Helicobacter pylori. Examples of Gram-positive bacteria include, but are not limited to, Staphylococcus aureus, Staphylococcus epidermidis, Streptococcus agalactiae, Group A Streptococcus, Streptococcus pyogenes, Enterococcus faecalis, Group B Gram-positive Streptococcus, Corynebacterium xerosis, and Listeria monocytogenes. Examples of fungi include yeasts such as Candida albicans. Examples of viruses include measles virus, herpes simplex virus (HSV-1 and -2), herpes family members (HIV, hepatitis C, vesicular stomatitis virus (VSV), visna virus, and cytomegalovirus (CMV). Examples of protozoa include Giardia.

“Therapeutically effective amount” as used herein, refers to an amount of formulation, composition, or reagent in a pharmaceutically acceptable carrier or a physiologically acceptable salt of an active compound that is of sufficient quantity to ameliorate the undesirable state of the patient, animal, material, or object so treated. “Ameliorate” refers to a lessening of the detrimental effect of the disease state or disorder, or reduction in contamination, in the receiver of the treatment.

“Pharmaceutical agent or drug” as used herein, refers to a chemical compound or composition capable of inducing a desired therapeutic or prophylactic effect when properly administered to a subject.

“Pharmaceutically acceptable carrier” as used herein, refers to conventional pharmaceutical carriers useful in the methods disclosed herein. Remington's Pharmaceutical Sciences, by E. W. Martin, Mack Publishing Co., Easton, Pa., 15th Edition (1975), describes compositions and formulations suitable for pharmaceutical delivery of TCR peptides and additional pharmaceutical agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

As used herein, the term “specificity determinant(s)” refers to positively charged amino acid residue(s) (including, for example, lysine, arginine, or ornithine) in the non-polar face of AMPs that could decrease hemolytic activity/toxicity but increase or maintain the same level of antimicrobial activity, thus increasing the therapeutic index of the AMP.

Antimicrobial peptides (AMPs) of this disclosure have antimicrobial activity by themselves or when covalently conjugated or otherwise coupled or associated with another molecule, e.g., alkanoyl group, polyethylene glycol, an antibody, a small-molecule antibiotic, a specific bacterial cell-surface targeting molecule or a carrier protein such as bovine serum albumin, so long as the peptides are positioned such that they can come into contact with a cell or unit of the target microorganism. These peptides may be modified by methods known in the art provided that the antimicrobial activity is not destroyed or substantially compromised.

The peptides of this disclosure may be isolated or purified. These peptides may be synthetic and can be produced by peptide synthesis techniques or by recombinant expression technology as understood in the art. As used herein, the term “purified” can be understood in to refer to a state of enrichment or selective enrichment of a particular component relative to an earlier state of crudeness or constituency of another component. This term can be considered to correspond to a material that is at least partially purified as opposed to a state of absolute purity. For example, a peptide composition may be considered purified even if the composition does not reach a level of one hundred percent purity with respect to other components in the composition.

Peptides as described above for use in accordance with the invention may be prepared by conventional modes of synthesis including genetic or chemical means.

Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). Chemical synthesis may be performed by methods well known in the art involving cyclic sets of reactions of selective deprotection of the functional groups of a terminal amino acid and coupling of selectively protected amino acid residues, followed finally by complete deprotection of all functional groups.

Synthesis may be performed in solution or on a solid support using suitable solid phases known in the art (for example by the methodology described in the Examples section of this disclosure, particularly Example 1) W. C. Chan and P. D. White; Fmoc Solid Phase Peptide Synthesis: 2) A Practical Approach (Practical Approach Series), Oxford University Press, U.S.A.; Chemistry of Peptide Synthesis by N. Leo Benoiton; CRC Press 2005; ISBN 9781574444544; 3) Tsuda, Y. and Okada, Y. (2010) Solution-Phase Peptide Synthesis, in Amino Acids, Peptides and Proteins in Organic Chemistry: Building Blocks, Catalysis and Coupling Chemistry, Volume 3 (ed A. B. Hughes), Wiley-VCH Verlag GmbH & Co. KGaA, Weinheim, Germany. doi: 10.1002/9783527631803.ch6; 4) Solid-phase peptide synthesis: A practical approach, E. Atherton, R. A Sheppard, Oxford university press 1989).

Since the peptides of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

In an alternative embodiment a peptide of the invention may be produced from or delivered in the form of a polynucleotide which encodes, and is capable of expressing, it. Such polynucleotides can be synthesised according to methods well known in the art, as described by way of example in Sambrook et al (1989, Molecular Cloning—a laboratory manual; Cold Spring Harbor Press). Such polynucleotides may be used in vitro or in vivo in the production of a peptide of the invention. Such polynucleotides may therefore be administered or used in the treatment of a microbial infection or another disease or condition as described herein.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individually, or in any combination. Additionally, unless otherwise specified, all isotopic variants of compounds disclosed herein are intended to be encompassed by the disclosure. As a brief illustration, it will be understood that any one or more hydrogens in a molecule disclosed can be replaced with deuterium or tritium.

Isotopic variants of a molecule are generally useful as standards in assays for the molecule and in chemical and biological research related to the molecule or its use. Methods for making such isotopic variants are known in the art.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, although the invention has been disclosed by various aspects that may include preferred embodiments and aspects, modifications and variations of the concepts herein disclosed may be resorted to by those skilled in the art. Such modifications and variations are considered to be within the scope of this invention as defined by the appended claims.

The peptides described herein can be used to prepare therapeutic pharmaceutical compositions, for example, by combining the compounds with a pharmaceutically acceptable diluent, excipient, or carrier. The peptides may be added to a carrier in the form of a salt or solvate. For example, in cases where peptides are sufficiently basic or acidic to form stable non-toxic acid or base salts, administration of the peptides as salts may be appropriate. Examples of pharmaceutically acceptable salts are organic acid addition salts formed with acids that form a physiological acceptable anion, for example, tosylate, methanesulfonate. acetate, citrate, malonate, tartrate, succinate, benzoate, ascorbate, a-ketoglutarate, and 3-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

Compositions of this Disclosure

When employed as pharmaceuticals, especially as antimicrobial agents administered to mammals, the AMPs of this disclosure are administered in the form of pharmaceutical compositions. These compounds can be administered by a variety of routes including oral, rectal, transdermal, subcutaneous, intravenous, intramuscular, intrathecal, and intranasal. Such pharmaceutical compositions are prepared in a manner well known in the pharmaceutical art and comprise at least one AMP of this disclosure.

The pharmaceutical compositions of the present invention contain, as the active ingredient, one or more of the AMPs of this disclosure, associated with pharmaceutically acceptable formulations. In making the compositions of this invention, the active ingredient is usually mixed with an excipient, diluted by an excipient or enclosed within a carrier which can be in the form of a capsule, sachet, paper or other container. An excipient is usually an inert substance that forms a vehicle for a drug. When the excipient serves as a diluent, it can be a solid, semi-solid, or liquid material, which acts as a vehicle, carrier or medium for the active ingredient. Thus, the compositions can be in the form of solutions, syrups, aerosols (as a solid or in a liquid medium), ointments containing, for example, up to 30% by weight of the active compound, soft and hard gelatin capsules, suppositories, sterile injectable solutions, and sterile packaged powders.

In preparing a formulation, it may be necessary to mill active compounds of this disclosure to provide the appropriate particle size prior to combining with the other ingredients. If the antimicrobial peptide is substantially insoluble, it ordinarily is milled to a particle size of less than 200 mesh. If the compound(s) is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, gum Arabic, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of this disclosure can be formulated to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a peptide of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 500 mg of the active ingredient of the present invention.

Formulations of this disclosure suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of this disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the compounds of this disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of this disclosure for rectal or vaginal administration may be presented as a suppository, which may be prepared by mixing one or more compounds of this disclosure with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of compounds of this disclosure to the body. Such dosage forms can be made by dissolving, dispersing or otherwise incorporating one or more compounds of this disclosure in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations include those suitable for administration by inhalation or insufflation or for nasal or intraocular administration. For administration to the upper (nasal) or lower respiratory tract by inhalation, the compounds of this disclosure are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of this disclosure and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of this disclosure may be administered by means of nose drops or a liquid spray, such as by means of a plastic bottle atomizer or metered-dose inhaler. Typical of atomizers are the Mistometer (Wintrop) and Medihaler (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administrations comprise one or more compounds of this disclosure in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

Suitable alkalinizing agents include alkali metal salts and alkaline earth metal salts. The alkali metal salts include sodium carbonate, sodium hydroxide, sodium silicate, disodium hydrogen orthophosphate, sodium aluminate, and other suitable alkali metal salts or mixtures thereof. Suitable alkaline metal salts include calcium carbonate, calcium hydroxide, magnesium carbonate, magnesium hydroxide, magnesium silicate, magnesium aluminate, aluminum magnesium hydroxide or mixture thereof. More particularly, calcium carbonate, potassium bicarbonate, calcium hydroxide, and/or sodium carbonate may be used as alkalinizing agents to obtain a formulation pH within the desired pH range of pH 8 to pH 13. The concentration of the alkalinizing agent is selected to obtain the desired pH, varying from about 0.1% to about 30%, by weight, and more preferably from about 12.5% to about 30%, by weight, of the total weight of the dosage formulation.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

### Methods for Preventing and Treating Microbial Infection

Another aspect of this disclosure provides methods for preventing and treating a microbial infection. These methods include administering to a subject in need thereof a therapeutically effective amount of a peptide or composition of this disclosure that kills or inhibits the growth of infectious microbes, thereby inhibiting or treating the microbial infections. The infecting microorganism may include Gram-negative bacteria.

The infecting microorganism may be a Gram-negative bacteria, which may include, but is not limited to, Escherichia coli, Pseudomonas aeruginosa, Acinetobacter baumannii, Salmonella spp Haemophilus influenzae, Neisseria spp Vibrio cholerae, Vibrio parahaemolyticus and Helicobacter pylori.

The antimicrobial peptides administered, preferably as a component of a pharmaceutical composition, can include a single antimicrobial peptide of this disclosure, or multiple peptides of this disclosure. The peptides may include peptides having at least 85%, or at least 90%, or at least 95% homology to a peptide sequence of SEQ ID NOs: 1, 3-8 and 10-17, and which effectively treat or prevent a microbial infection. The peptides may include fragments of the peptides of SEQ ID NOs: 1, 3-8 and 10-17 that retain the ability to effectively treat or prevent a microbial infection. An exemplary peptide includes the amino acid sequence set forth in SEQ ID NO:3. Appropriate peptides to use in the methods disclosed herein can be determined by those skilled in the art.

Therapeutic AMPs of this disclosure may be administered by a number of routes, including orally, topically, and by parenteral administration, including for example, intravenous infusion or injection, intraperitoneal, intramuscular, intradermal, intrathecal, intrasternal, or intraarticular injection. One of skill in the art can readily determine the appropriate route of administration.

The therapeutically effective amounts of the AMPs of this disclosure that inhibit or kill an infecting microorganism will depend upon the subject being treated, the severity and type of the infection, and the manner of administration. For example, a therapeutically effective amount of a peptide of this disclosure can vary from about 1 mcg/injection up to about 10 mg/injection. The exact amount of the peptide is readily determined by one of skill in the art based on the age, weight, sex, and physiological condition of the subject. Effective doses can be extrapolated from dose-response curves derived from in vitro or animal model test systems.

One or more peptides of this disclosure that effectively inhibit or kill an infecting microorganism can be administered in conjunction with one or more additional pharmaceutical agents. The additional pharmaceutical agents can be administered at the same time as, or sequentially with, the peptide(s) of this disclosure. The additional pharmaceutical agent may be an additional antimicrobial agent. When administered at the same time, the additional pharmaceutical agent(s) can be formulated in the same composition that includes the peptide(s) of this disclosure.

In one embodiment, the invention provides a product comprising a peptide of the invention and at least one other therapeutic agent as a combined preparation for simultaneous, separate or sequential use in therapy. In one embodiment, the therapy is the treatment of a microbial infection. Products provided as a combined preparation include a composition comprising the peptide of the invention and the other therapeutic agent(s) together in the same pharmaceutical composition, or the agent of the invention and the other therapeutic agent(s) in separate form, e.g. in the form of a kit.

In the combination therapies of the invention, the peptide of the invention and the other therapeutic agent may be manufactured and/or formulated by the same or different manufacturers. Moreover, the peptide of the invention and the other therapeutic may be brought together into a combination therapy: (i) prior to release of the combination product to physicians (e.g. in the case of a kit comprising the peptide of the invention and the other therapeutic agent); (ii) by the physician themselves (or under the guidance of the physician) shortly before administration; (iii) in the patient themselves, e.g. during sequential administration of the peptide of the invention and the other therapeutic agent.

Accordingly, the invention provides the use of peptide of the invention for treating a microbial infection, wherein the medicament is prepared for administration with another therapeutic agent. The invention also provides the use of another therapeutic agent for treating a microbial infection, wherein the medicament is administered with a peptide of the invention.

The invention also provides a peptide of the invention for use in a method of treating a microbial infection, wherein the peptide of the invention is prepared for administration with another therapeutic agent. The invention also provides another therapeutic agent for use in a method of treating a microbial infection, wherein the other therapeutic agent is prepared for administration with a peptide of the invention. The invention also provides a peptide of the invention for use in a method of treating a microbial infection, wherein the peptide of the invention is administered with another therapeutic agent. The invention also provides another therapeutic agent for use in a method of treating a microbial infection, wherein the other therapeutic agent is administered with a peptide of the invention.

The invention also provides the use of a peptide of the invention for treating a microbial infection, wherein the subject has previously (e.g. within 24 hours) been treated with another therapeutic agent. The invention also provides the use of another therapeutic agent for treating a microbial infection, wherein the subject has previously (e.g. within 24 hours) been treated with a peptide of the invention.

Compositions may additionally comprise molecules which assist or augment the action of the agents of the invention, e.g. an antibiotic, which inhibits bacterial or fungal growth or kills bacteria or fungi. A peptide of the invention may be combined in variable or fixed ratio combinations with antibiotics from any known antibiotic class. Specifically, these antibiotics may include antibiotics of the lincomycin family (a class of antibiotic agents originally recovered from Streptomyces lincolnensis); antibiotics of the tetracycline family (a class of antibiotic agents originally recovered from Streptomyces aureofaciens); and sulfur-based antibiotics such as the sulfonamides. Beta-lactams that can be combined include penems, carbapenems (imipenem, meropenem, ertapenem, doripenem, panipenem, biapenem, and the like), monobactams (aztreonam, tigimonam, carumonam, BAL30072, and the like), as well as a variety of other beta-lactam cell envelope antibiotics (see: https://en.wikipedia.org/wiki/Monobactam

Specific examples of some suitable antibiotics of the lincomycin family include lincomycin, clindamycin, and clindamycin phosphate.

Specific examples of macrolide antibiotics include erythromycin, azithromycin, clarithromycin, dirithromycin, roxithromycin, carbomycin A, josamycin, kitasamycin, midecamycin/midecamycin acetate and troleandomycin.

Specific examples of ketolide antibiotics include telithromycin, cethromycin, solithromycin, spiramycin, ansamycin, oleandomycin, carbomycin, and tylosin.

Specific examples of antibiotics of the tetracycline family include tetracycline itself, chlortetracycline, oxytetracycline, demeclocycline, rolitetracycline, methacycline and doxycycline.

Specific examples of sulfur-based antibiotics include the sulfonamides, sulfacetamide, sulfabenzamide, sulfadiazine, sulfadoxine, sulfamerazine, sulfamethazine, sulfamethizole, sulfisoxazole, and sulfamethoxazole.

Further examples of combinable antibiotics include the oxazolidinones such as zyvox (linezolid), peptide antibiotics such as the polymixins, quinolones, fluoroquinolones (https://en.wikipedia.orgiwiki/Quinolone_antibiotic), aminoglycosides (https://en.wikipedia.org/wiki/Aminoglycoside), and rifamycins (https://en.wikipedia.org/wiki/Rifamycin).

Combinable antibiotics can also include various antibacterial agents, antifungal agents, antimycotic agents and antiviral agents; penicillins such as ampicillin or amoxicillin, cephalosporins such as cephalothin and ceclor (cephachlor), aminoglycosides such as, kanamycin, macrolides such as erythromycin, nystatin, and amphotericin; and the antibiotics amikacin, bacillomycin, chloramphenicol, doxorubicin, doxycycline, ethambutol, gentamicin, isoniazid, kanamycin, carbacephalosporins such as lorabid (loracarbef), mupirocin, neomycin, pyrrolnitrin, rifampin, streptomycin, and vancomycin.

Those skilled in the art can determine an appropriate time and duration of therapy that includes the administration of a peptide of this disclosure to achieve the desired preventative or ameliorative effects on the subject treated.

The invention now being generally described will be more readily understood by reference to the following examples, which are included merely for the purposes of illustration of certain aspects of the invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Peptide Design, Specificity Determinants and Amphipathicity

Enantiomeric forms of AMPS with all-D-amino acids have shown equal activities to their all-L-enantiomers. The advantage of all-D-peptides is that they are resistant to proteolytic enzyme degradation, which enhances their potential as therapeutic agents. In these studies, the inventors de novo designed, synthesized, purified and characterized twelve all-D amphipathic alpha-helical antimicrobial peptides with “specificity determinants,” denoted D26, D51, D52, D53, D54, and D55 (sequences shown in Table 1A), D56, D57 and D58 (sequences shown in Table 1B) and D59, D60 and D61 (sequences shown in Table 1C). In addition, the sequence of one peptide without specificity determinants D26(K13A,K16A) is shown in Table 1A.

All peptides were prepared in an analogous manner using the following procedures

| D-number | Sequence |
|---|---|
| D26 | Ac-KLKSLLSTLSSAKKKKLSTLLSALSK-NH₂ |
| D51 | Ac-ALKKLLSTLSSAKKKKLSTLLSALSK-NH₂ |
| D52 | Ac-ALKKLLSTLSSAKSSKLSTLLKALKK-NH₂ |
| D53 | Ac-ALSSLLKKLSSAKSSKLSTLLKALKK-NH₂ |
| D54 | Ac-ALSSLLSTLKKAKSSKLSTLLKALKK-NH₂ |
| D55 | Ac-ALSSLLSTLKKAKSSKLKKLLKALSS-NH₂ |
| D56 | Ac-ALKKLLSTLSSA(*Orn)SS(*Orn)LSTLLKALKK-NH₂ |
| D57 | Ac-ALKKLLSTLSSA(*DBu)SS(*DBu)LSTLLKALKK-NH₂ |
| D58 | Ac-ALKKLLSTLSSA(*Arg)SS(*Arg)LSTLLKALKK-NH₂ |
| D59 | Ac-AL(*Orn)(*Orn)LLSTLSSAKSSKLSTLL(*Orn)AL(*Orn)(*Orn)-NH₂ |
| D60 | Ac-AL(*DBu)(*DBu)LLSTLSSAKSSKLSTLL(*DBu)AL(*DBu)(*DBu)- |
| | NH₂ |
| D61 | Ac-AL(*Arg)(*Arg)LLSTLSSAKSSKLSTLL(*Arg)AL(*Arg)(*Arg)-NH₂ |
| D26 | Ac-KLKSLLSTLSSAAKKALSTLLSALSK-NH₂ |
| (K13A, | |
| K16A) | |

### Solid-Phase Peptide Synthesis:

Standard solid-phase peptide synthesis methodology was conducted using 9-fluorenylmethoxycarbonyl (Fmoc) chemistry and 4-Rinkamide MBHA resin (P3 Biosystems, Louisville, Ky.) or Rink Amide-ChemMatrix® resin (Biotage, Charlotte, N.C.) using a Focus-XC peptide synthesizer (Aapptec, Louisville, Ky.). The coupling procedure used (Benzotriazol-1-yloxy)tris(dimethylamino) phosphonium hexafluorophosphate (Bop)/1-hydroxybenzotriazole (HOBt) in dimethylformamide (DMF) with N,N-diisopropylethylamine (DIPEA) in N-methyl-2-pyrrolidinone (NMP) with the first coupling at room temperature for one hour and the second coupling at 50° C. for another hour. The deprotection procedure (removal of Fmoc protecting group) was carried out by treatment of the resin with 0.1 M HOBt in DMF with 20% piperidine. After completion of the synthesis of the desired amino-acid sequence the N-terminus protecting group was removed as described above and the free amine was acetylated with acetic anhydride (Ac₂O) following the procedure described below.

Acetylation of N-Terminus-NH₂

The N-terminal free amino peptide-Rink Amide MBHA resin (0.2 mmol) was suspended in DMF (5 mL) and a solution of Ac₂O (0.95 mL, 10 mmol) and DIPEA (1.74 mL, 10 mmol) in DMF (5 mL) was added. The sample was mixed for 2 h and the acetylation procedure repeated until the Kaiser test (Ninhydrin Test) was negative. The resin was washed (DMF×5) and dried by lyophilisation overnight.

Global Deprotection and Cleavage from Resin

The peptide was cleaved from the peptide resin from above with a mixture of 90% trifluoroacetic acid (TFA), 5% water and 5% triisopropylsilane (TIS) for 1-2 h. The resin was removed by filtration and the peptide precipitated with ice-cooled diethyl ether on ice for 1-2 h. The pellet was concentrated under reduced pressure, re-dissolved in acetonitrile/water (1:1, with 0.2% TFA) and the solution lyophilized to obtain the crude peptide which was purified by HPLC as described below.

Analytical and Preparative Purification by Reversed-Phase Chromatography

Analytical RP-HPLC:

Column, Luna C18 (2), 250×4.6 mm I.D., 5 μm particle size, 100 Å pore size from Phenomenex. Run conditions: linear AB gradient (1% acetonitrile/min, starting from 2% acetonitrile) at a flow-rate of 1 mL/min, where eluent A is 0.2% aq. TFA and eluent B is 0.18% TFA in acetonitrile; temperature, 30° C.

Preparative RP-HPLC:

Column, Luna C18 (2), 250×30 mm I.D., 10 um particle size, 100 Å pore size from Phenomenex. Peptides were dissolved in 0.2% aq. TFA containing 2% acetonitrile to a final concentration of 10 mg/mL, filtered sequentially through a 0.45 μm then 0.22 μm Millipore filters, and loaded onto the column via multiple 20-mL injections into a 20-mL injection loop at a flow-rate of 10 mL/min. Run conditions: 1% acetonitrile/min gradient up to an acetonitrile concentration 15% below that required to elute the peptide during analytical RP-HPLC on the same packing material, then shallow gradient elution (0.1% acetonitrile/min) at a flow-rate of 10 mL/min (same eluents as shown above for analytical RP-HPLC) at room temperature. The preparative HPLC instrumentation used was obtained from Aapptec, Louisville, Ky. consisting of two P300 pumps for preparing gradients with flow rates up to 50 mL/min and a variable UV/VIS wavelength detector (UV 3000) connected to a LKB fraction collector. Fractions were analyzed by LC/MS using a Agilent 1100 series HPLC with autosampler connected to an Agilent LC/MSD Trap XT (Agilent Technologies, Inc., Santa Clara, Calif.). The column used was a HALO 2-C18 column 2.1×50 mm, 2μ particle size from Advanced Materials Technology (Wilmington, DC). Solid-core particles with 0.4-micron thick porous shell and 90 Å pore size or an Agilent Zorbax 300SB-C8, 4.6 mm I.D.×100 mm, 300 Å pore size, 3.5 micron particle size.

Conversion to Final Acetate Salt of Peptides

The HPLC purified peptide trifluoroacetate (TFA) salt from above was dissolved in 95:5 acetic acid/H₂O (15 mL) and transferred to a polypropylene conical tube (50 mL) and allowed to stand for 20 minutes to enable counter ion exchange to occur. The sample was frozen (liquid N₂) and the peptide lyophilized overnight. This process was repeated twice and the resulting peptide acetate salt was suspended in H₂O (10 mL) and lyophylised overnight to give the desired compound as an acetate salt.

### Determination of Peptide Amphipathicity:

Amphipathicity of peptides at pH 7 and pH 2 was determined by the calculation of hydrophobic moment, using the software package EMBOSS 6.5.7 and the Hmoment application, modified to include hydrophobicity scales determined in the inventors' laboratory. The hydrophobicity scales used in this study are listed as follows: At pH 7, Trp, 33.0; Phe, 30.1; Leu, 24.6; Ile, 22.8; Met, 17.3; Tyr, 16.0; Val, 15.0; Pro, 10.4; Cys, 9.1; His, 4.7; Ala, 4.1; Thr, 4.1; Arg, 4.1; Gln, 1.6; Ser, 1.2; Asn, 1.0; Gly, 0.0; Glu, −0.4; Asp, −0.8 and Lys, −2.0 (polar face), Lys, −18.48 (center of non-polar face). These hydrophobicity coefficients were determined from RP-HPLC at pH 7 (10 mM PO₄ buffer containing 50 mM NaCl) of a model random coil peptide with a single substitution of all 20 naturally occurring amino acids. At pH 2, these coefficients were determined in 20 mM trifluoroacetic acid (TFA), Trp, 32.4; Phe, 29.1; Leu, 23.3; Ile, 21.4; Met, 15.7; Tyr, 14.7; Val, 13.4; Pro, 9.0; Cys, 7.6; Ala, 2.8; Glu, 2.8; Thr, 2.3; Asp, 1.6; Gln, 0.6; Ser, 0.0; Asn, −0.6; Gly, 0.0; Arg. 0.6; His, 0.0; Lys, 2.8 (polar face), Lys, -18.48 (center of non-polar face). These HPLC-derived scales reflect the relative difference in hydophilicity/hydrophobicity of the 20 amino acid side-chains more accurately than previously determined scales (see recent review where this scale was compared to other scales; Mant, C. T., et al. Biopolymers 2009, 92:573-95.). The hydrophobicity/hydrophilicity coefficients for Lys residues centered in the non-polar face at pH 2.0 and pH 7.0 were assigned values of −18.48 determined by reversed-phase chromatography of the identical peptides where Ala was substituted by Lys on the non-polar face at position 13 and 16. Position X was placed in the sequence where these values are to be used in the Hmoment calculations when Lys is in the center of the non-polar face.

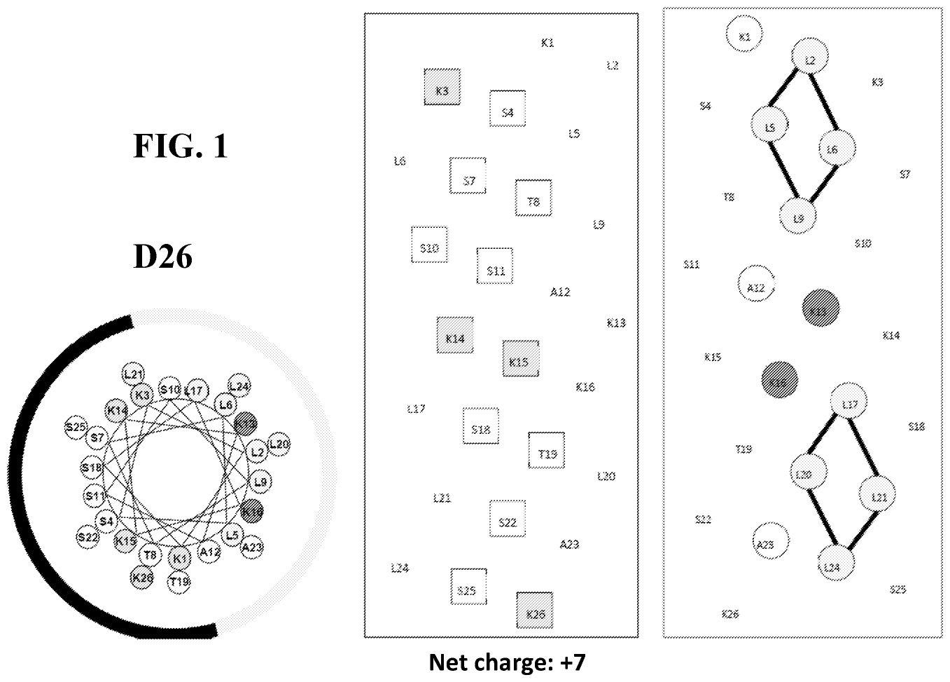

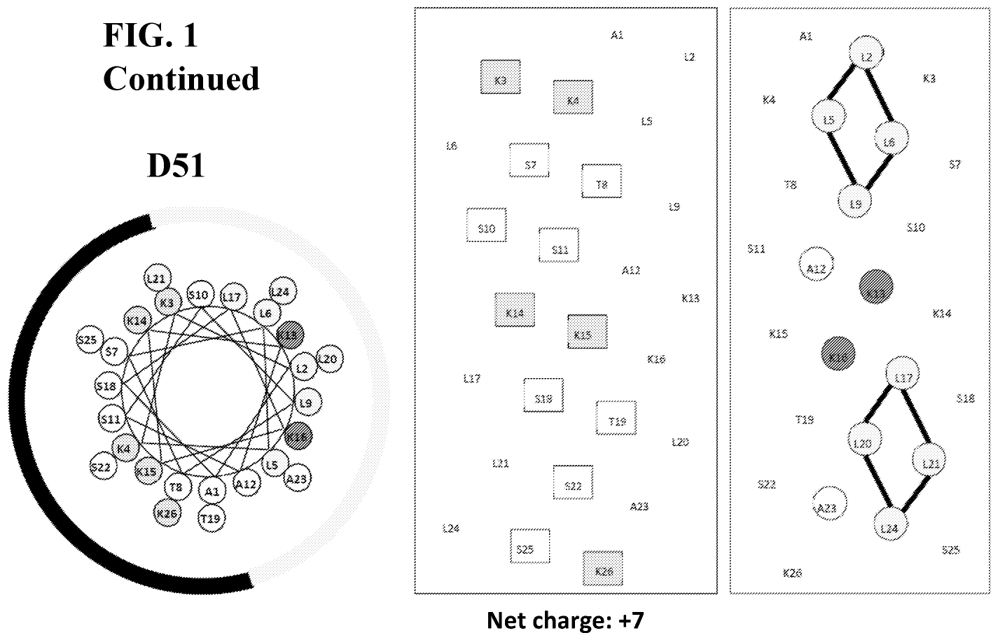

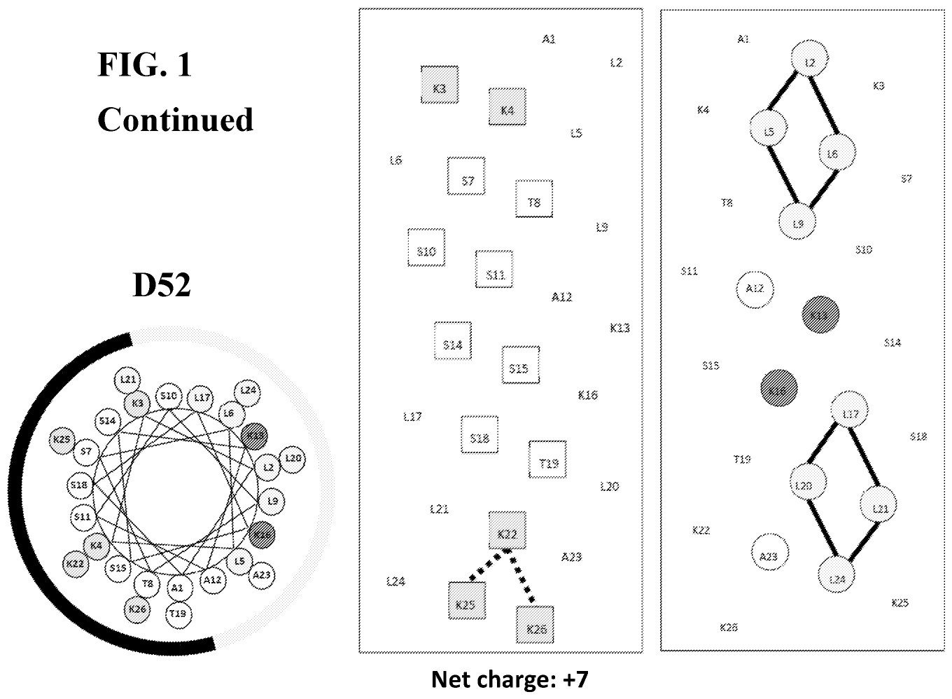

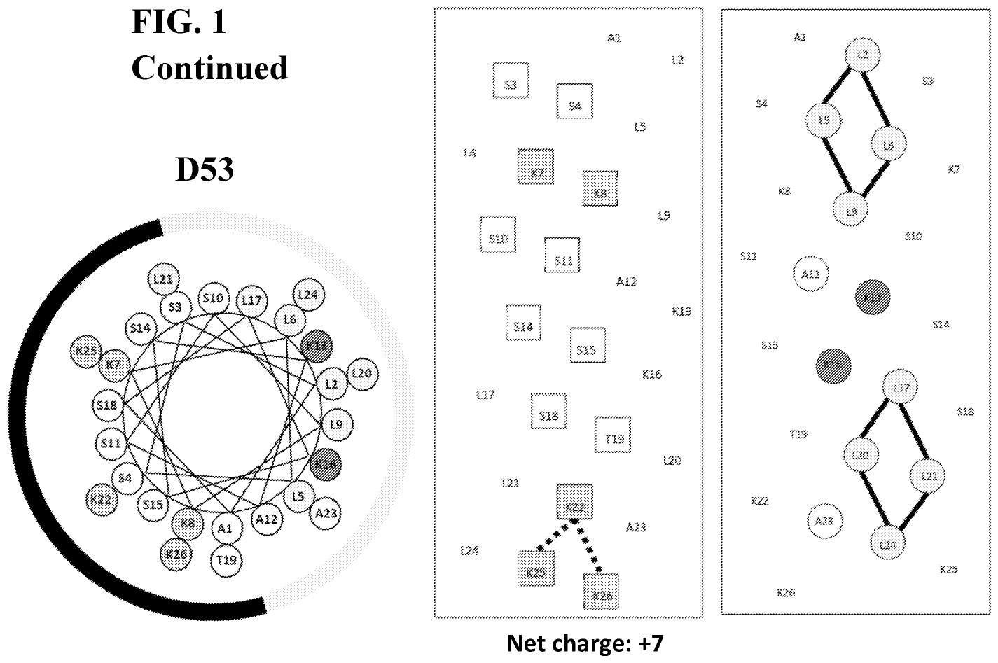

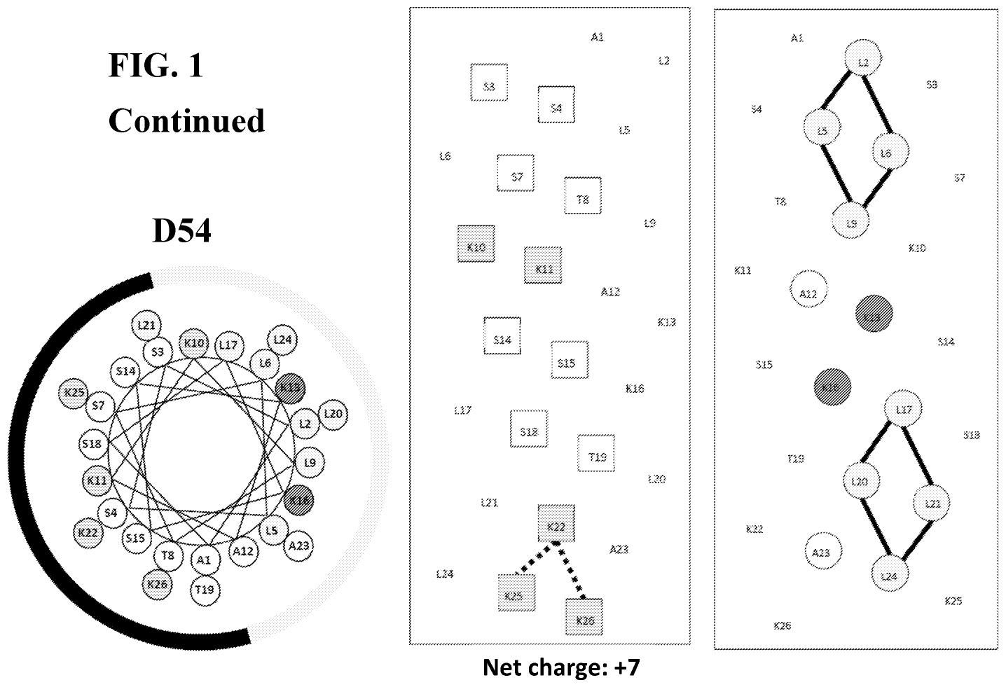

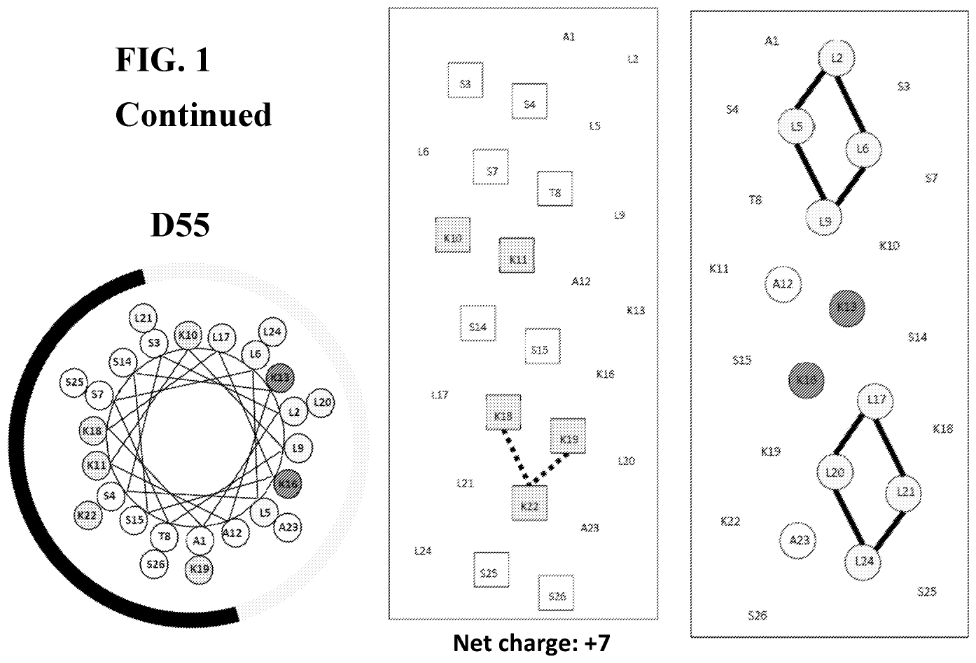

FIG. 1 shows the amino acid sequences in helical wheel and helical net representations. The inventors have displayed two versions of the helical nets wherein the polar residues are displayed along the center of the helical net (top) and where the non-polar residues are displayed along the center of the helical net (bottom). Peptides D26, D51, D52, D53, D54, and D55 are all very amphipathic alpha-helical peptides. These six peptides have a net positive charge of +7, and vary from one another by the arrangement of the five positively charged Lys residues on the polar face (FIG. 1). The hydrophobic/non-polar faces of these peptides are identical (FIG. 1). These representations in FIG. 1 allow easy comparison of different analogs and these sequence differences will be used to explain their biological and biophysical properties described below.

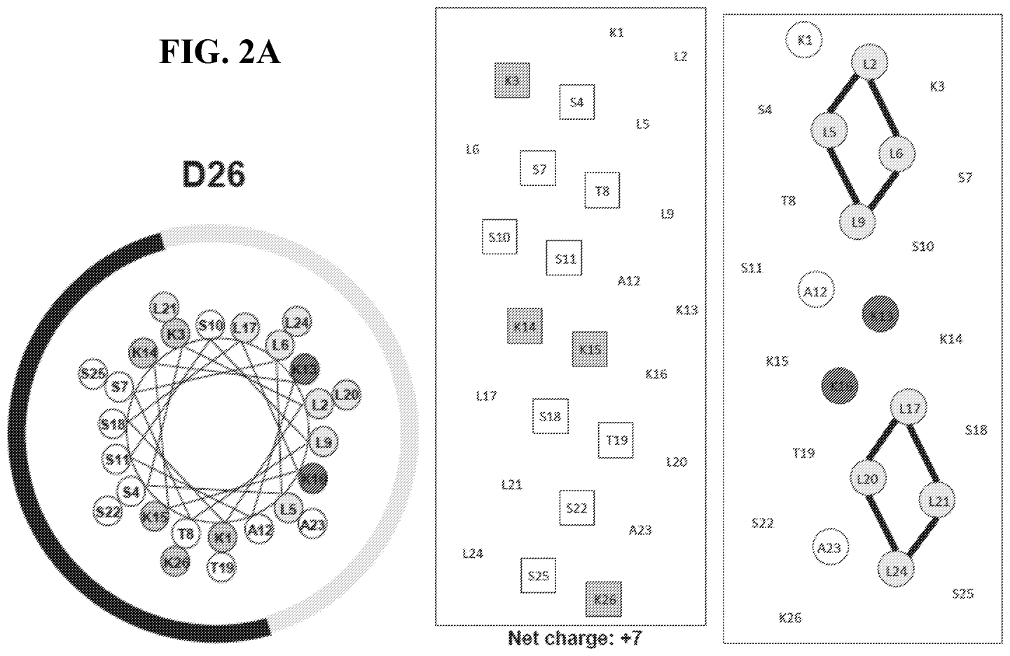

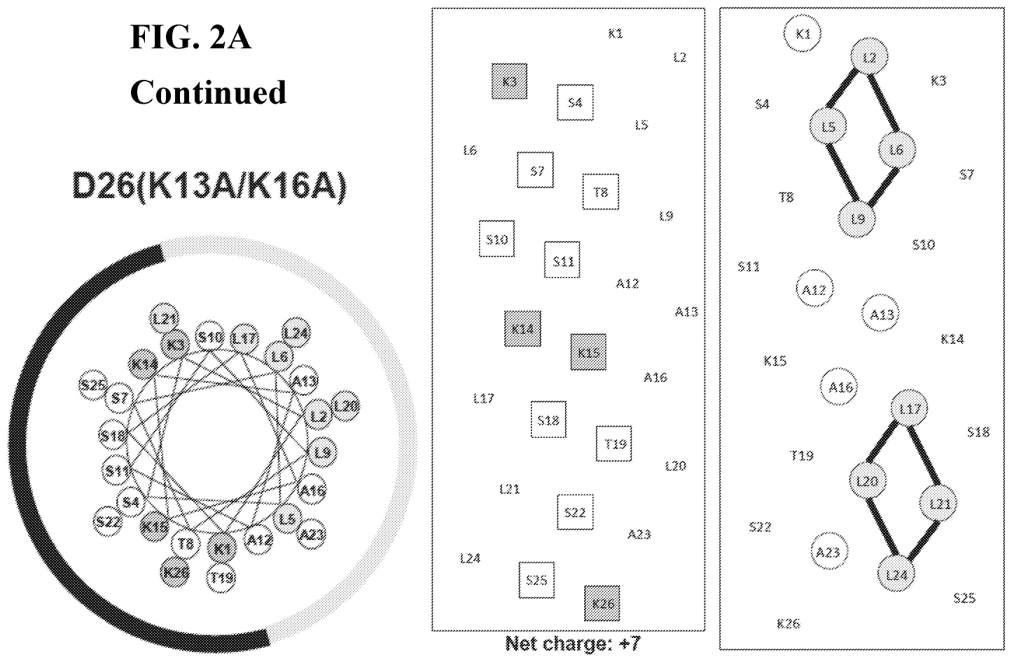

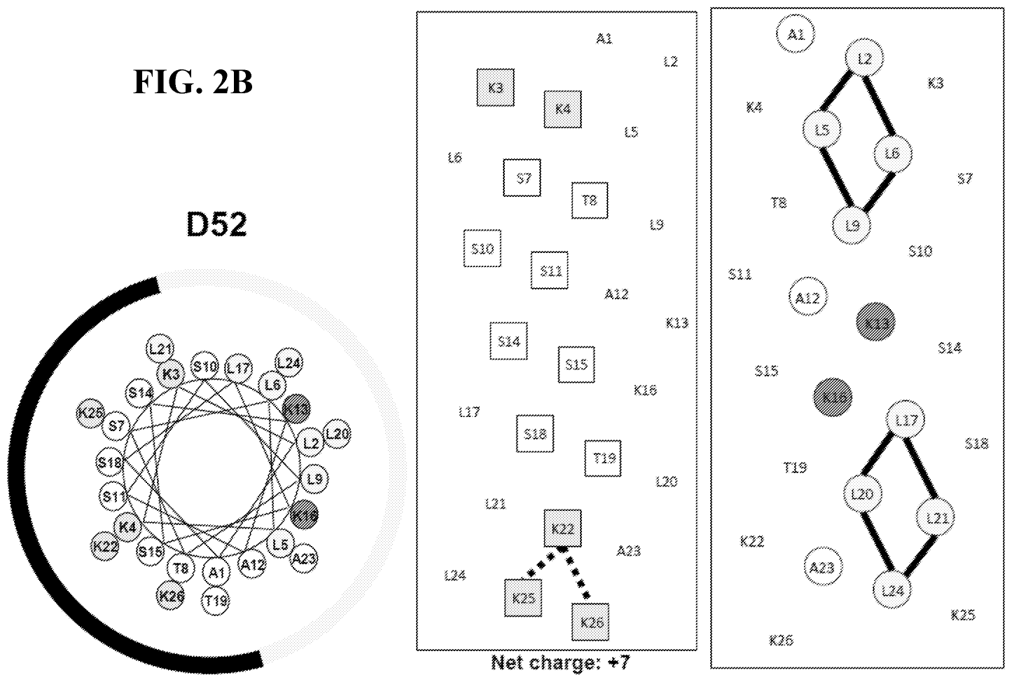

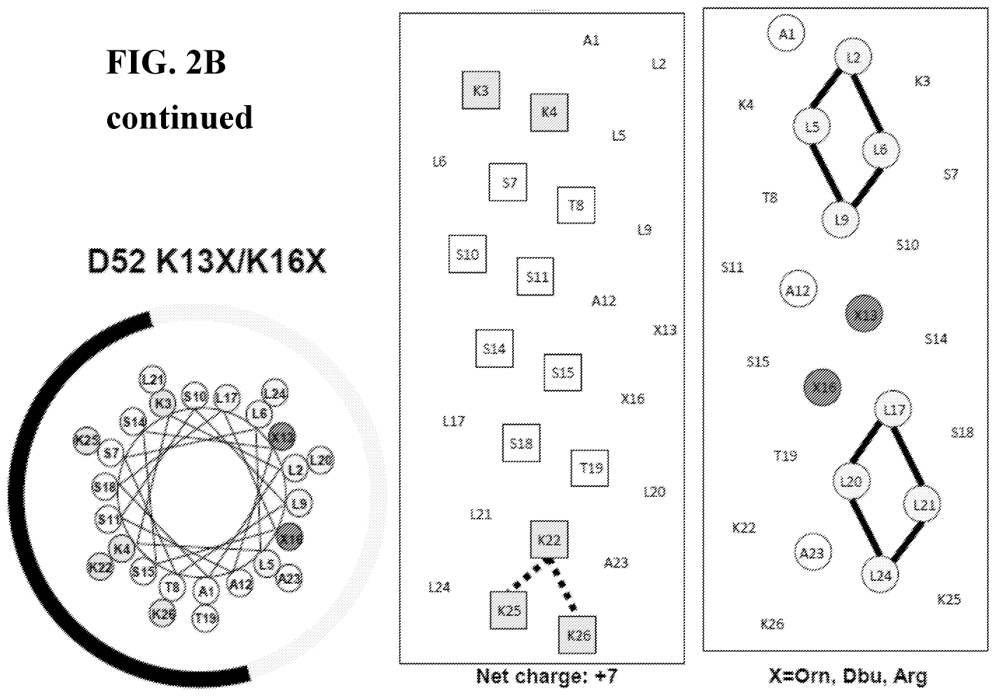

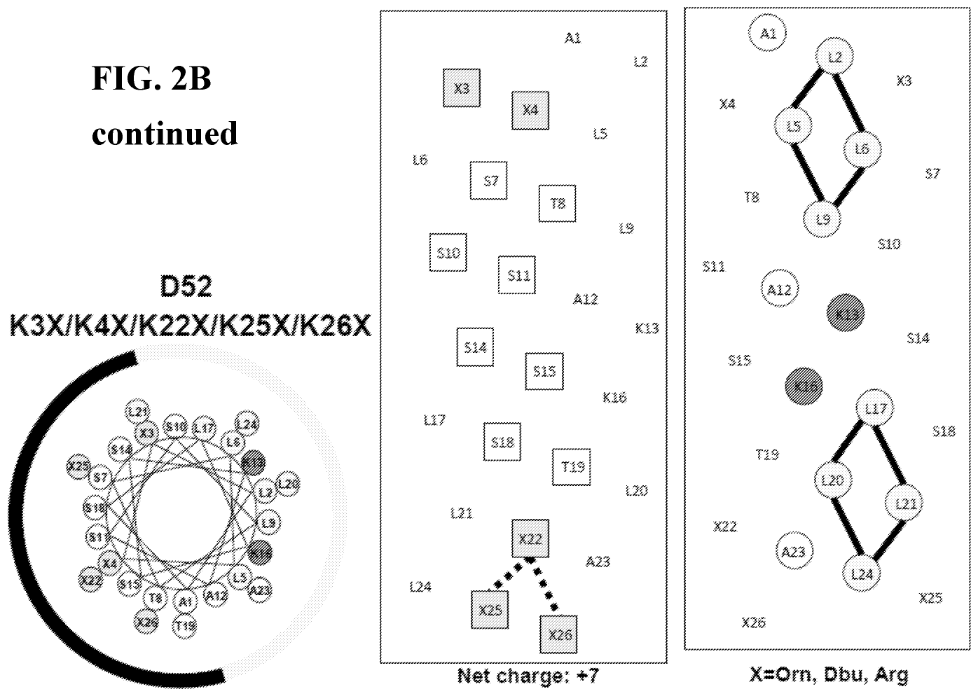

SEQ ID NO:3 (D26) contains two specificity determinants (K13 and K16) while SEQ ID NO:2 [D26 (K13A, K16A)] is without specificity determinants (K13A and K16A). This comparison shows that SEQ ID NO:3 (D26) has two specificity determinants on the non-polar face (K13 and K16) whereas in SEQ ID NO:2 [D26 (K13A, K16A)] alanine replaces the two lysine residues, and therefore this peptide has no specificity determinants. FIG. 2 shows the amino acid sequence of peptide SEQ ID NO:5 [D52 (with specificity determinants K13 and K16] compared to SEQ ID NO:10 [D52(K13X/K16X) where X is either Lys, Orn, Dbu or Arg]. Both specificity determinants are replaced at the same time (Table 1B). The third sequence shown in FIG. 2 is SEQ ID NO:14 [D52 (K3X/K4X/K22X/K25X/K26X where X is either Lys, Orn, Dbu or Arg]. As shown in Table 1C all 5 positions when changed are the same amino acid (5 Lys, 5 Orn, 5 Dbu or 5 Arg residues). These positions are all on the polar face of the amphipathic α-helix.

The design concept of “specificity determinants” (positively charged lysine residues in the center of the non-polar face of amphipathic alpha-helical AMPs) was introduced previously to achieve the following biophysical and biological properties: 1) disrupt the continuous hydrophobic surface that stabilizes the alpha-helical structure of the AMPs that lack “specificity determinants”; 2) reduce the hydrophobicity on the non-polar face and overall hydrophobicity as measured by retention time at 25° C. by reversed-phase chromatography (RP-HPLC); 3) dramatically reduce peptide self-association in aqueous conditions as measured by a novel procedure developed in the inventors' laboratory referred to as temperature profiling in RP-HPLC; 4) dramatically reduce toxicity to normal cells as measured by hemolytic activity to human red blood cells; 5) maintain or enhance antimicrobial activity; 6) dramatically improve the therapeutic indices of AMPs with specificity determinants compared to AMPs lacking these determinants; 7) the AMPs with specificity determinants encode selectivity for Gram-negative pathogens by significantly decreasing Gram-positive activity and hemolytic activity; 8) the AMPs are active against A. baumannii strains resistant to polymyxin B and polymyxin E (Colistin); 9) the specificity determinants allow the AMPs to discriminate between eukaryotic and prokaryotic cell membranes; 10) the specificity determinants ensure excellent antimicrobial activity in the presence of human serum. In the current study de novo designed AMPs were tested against seven diverse clinical isolates of the Gram-negative pathogen A. baumannii and seven A. baumannii strains resistant to polymyxin B and polymyxin E (Colistin). In addition, the inventors tested these AMPs against six diverse clinical isolates of the Gram-negative pathogen, P. aeruginosa, and nine Gram-positive methicillin-sensitive S. aureus clinical isolates and eight Gram-positive methicillin/oxacillin-resistant S. aureus strains. This testing allows for the determination of pathogen selectivity between Gram-negative and Gram-positive pathogens as the location of the positively charged residues on the polar face is varied.

### Example 2: Peptide Hydrophobicity

Retention behavior in RP-HPLC is an excellent method to represent overall peptide hydrophobicity. Retention times of peptides are highly sensitive to the conformational status of the peptides upon interaction with the hydrophobic environment of the column matrix. The non-polar faces of amphipathic alpha-helical and amphipathic cyclic beta-sheet peptides represent a preferred binding domain for interaction with the hydrophobic matrix of the reversed-phase column. In this study, the observed peptide hydrophobicity was determined by RP-HPLC retention time as described in the methods section and are relative hydrophobicities because they are dependent on the TFA concentration and organic solvent in the mobile phase, gradient rate, temperature, flow rate and the column used.

Analytical and Preparative Purification by Reversed-phase Chromatography: Analytical RP HPLC:

Column, Luna C18 (2), 250×4.6 mm I.D., 5 micrometer particle size, 100 Å pore size from Phenomenex. Run conditions: linear AB gradient (1% acetonitrile/min, starting from 2% acetonitrile) at a flow-rate of 1 ml/min, where eluent A is 0.2% aq. TFA and eluent B is 0.18% TFA in acetonitrile; temperature, 30° C. Preparative RP-HPLC:

Peptides were dissolved in 0.2% aq. TFA containing 2% acetonitrile to a final concentration of 10 mg/ml. Following filtration through a 0.45 micrometer Millipore filter and subsequently through a 0.22 μm filter, the peptide solutions were loaded onto the column via multiple 20-ml injections into a 20-ml injection loop at a flow-rate of 5 ml/min. Column, Luna C18 (2), 250×30 mm I.D., 10 um particle size, 100 Å pore size from Phenomenex. Run conditions: 2% acetonitrile/min gradient up to an acetonitrile concentration 15% below that required to elute the peptide during analytical RP-HPLC, then shallow gradient elution (0.1% acetonitrile/min) at a flow-rate of 10 ml/min (same eluents as shown above for analytical RP-HPLC); room temperature.

### Example 3: Peptide Secondary Structure

Characterization of Helical Structure:

The mean residue molar ellipticities of peptides were determined by circular dichroism (CD) spectroscopy, using a Jasco J-815 spectropolarimeter (Jasco Inc. Easton, Md., USA) at 5° C. under benign (non-denaturing) conditions (50 mM NaH₂PO₄/Na₂HPO₄/100 mM KCl, pH 7.0), hereafter referred to as benign buffer, as well as in the presence of an alpha-helix inducing solvent, 2,2,2-trifluoroethanol, TFE, (50 mM NaH₂PO₄/Na₂HPO₄/100 mM KCl, pH 7.0 buffer/50% TFE). A 10-fold dilution of an approximately 500 microM stock solution of the peptide analogs was loaded into a 0.1 cm quartz cell and its ellipticity scanned from 195 to 250 nm. Peptide concentrations were determined by amino acid analysis.

Circular Dichroism spectroscopy (CD) Results

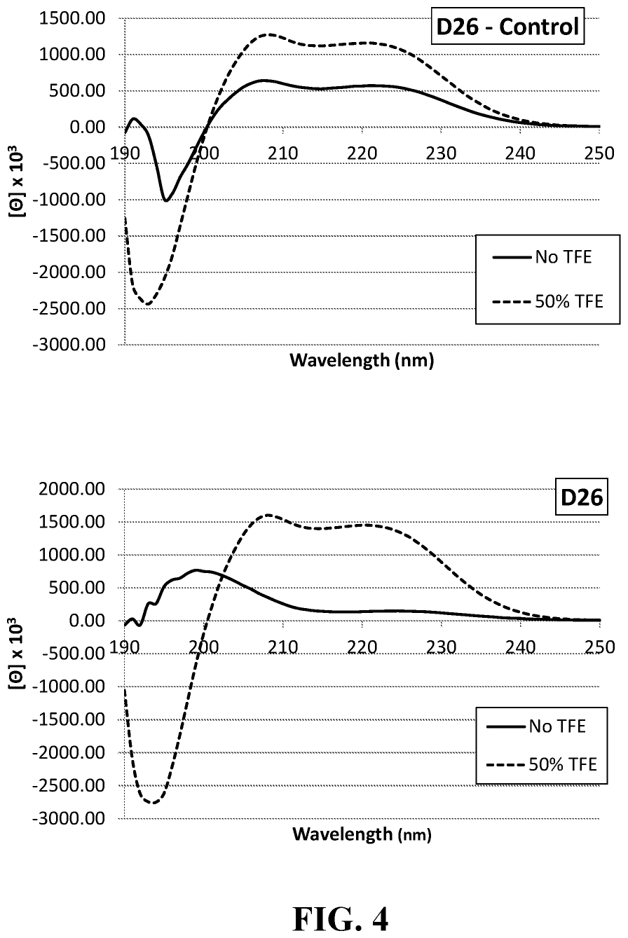

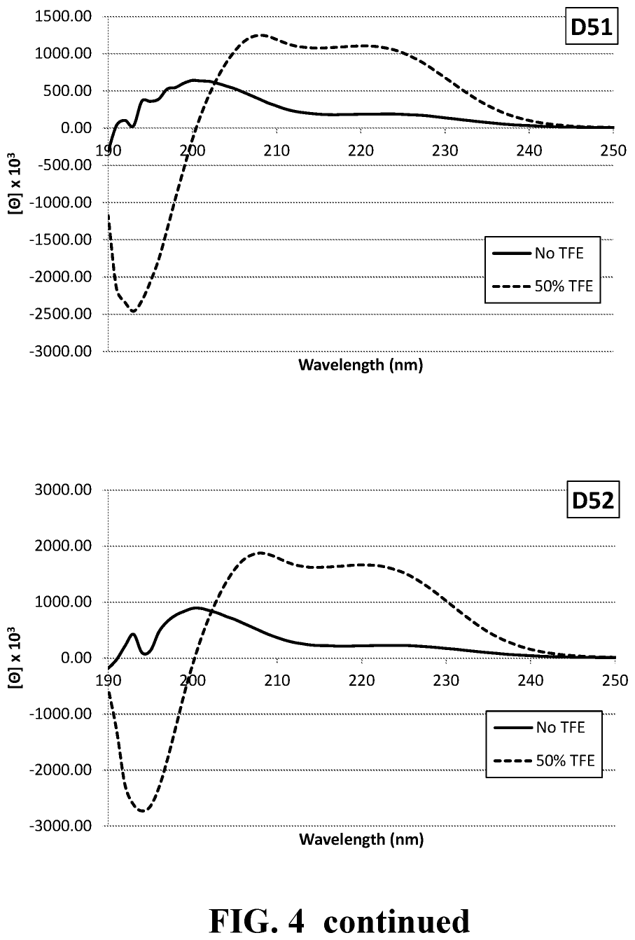

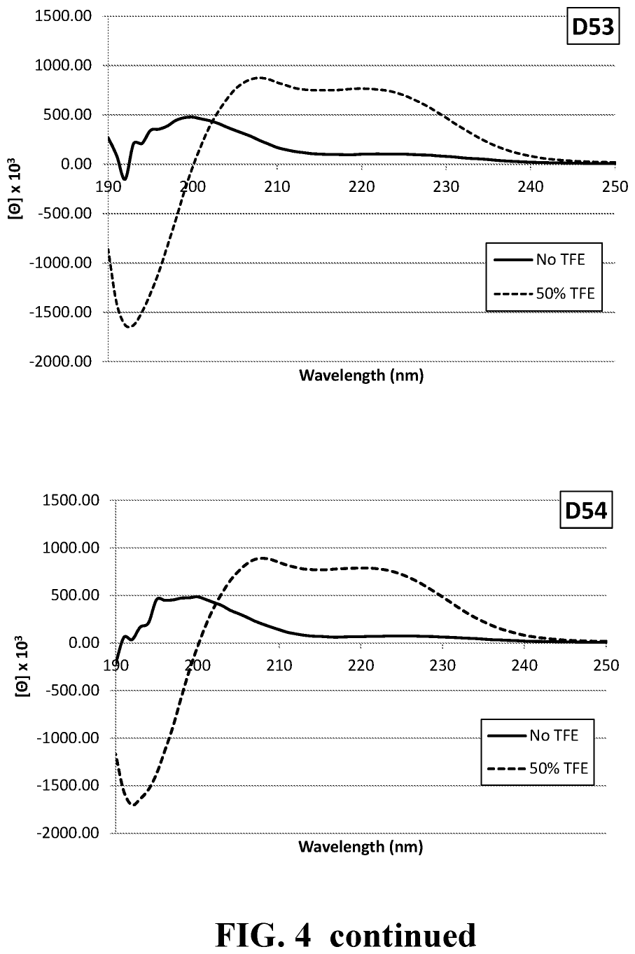

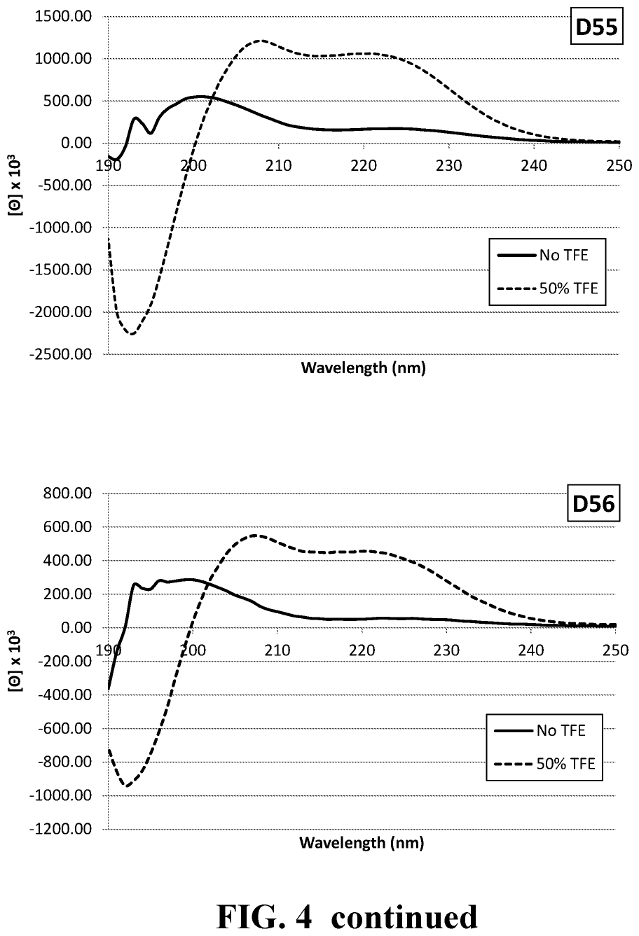

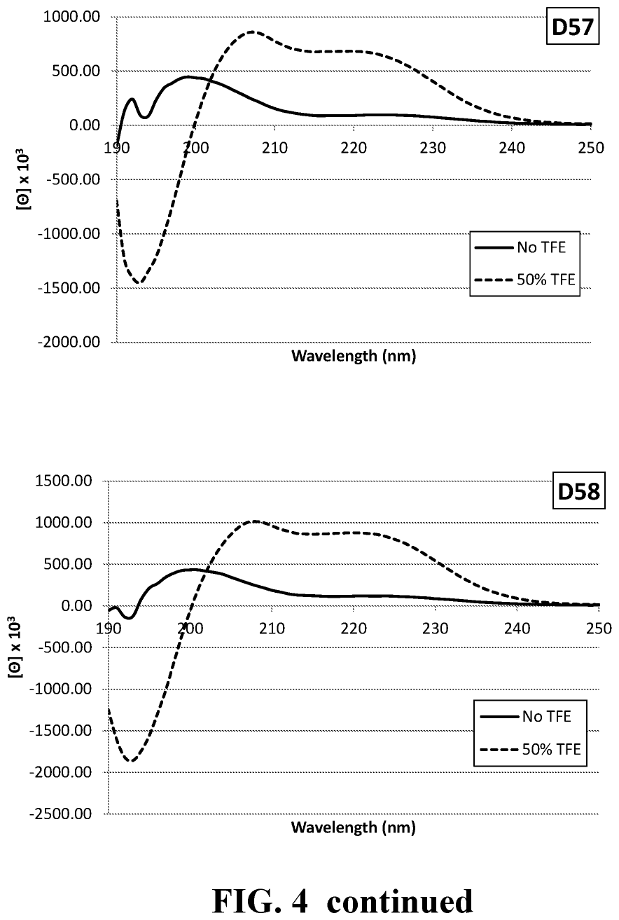

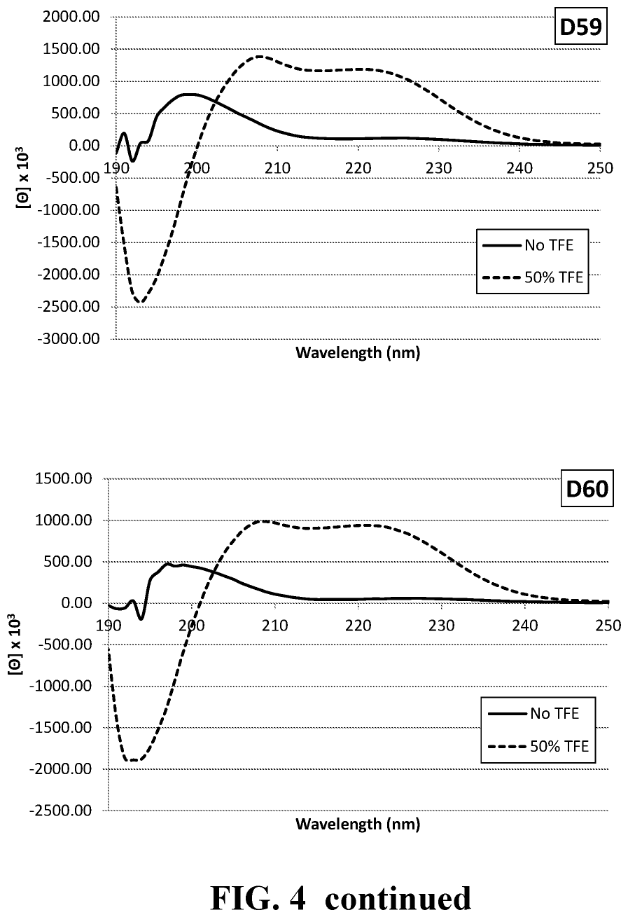

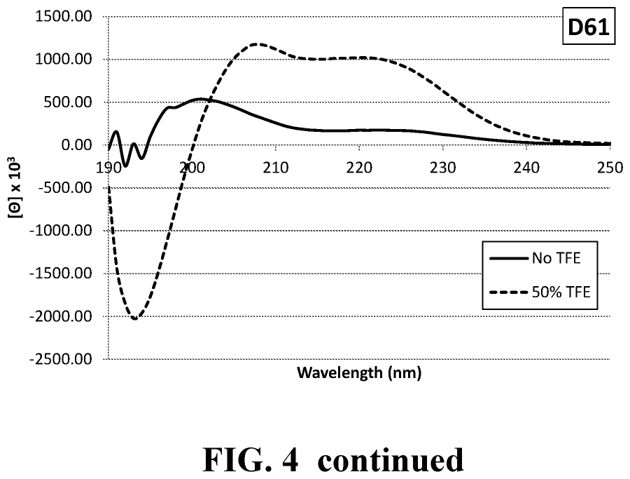

SEQ ID NO:2 [D26 (K13A/K16A)], which does not have specificity determinants at positions 13 and 16 on the non-polar face, was used as a control. The data clearly shows that this D-peptide is α-helical in aqueous buffer conditions (50 mM potassium phosphate buffer, pH 7.0 containing 100 mM KCl) and that significant α-helix is induced when placing the peptide in a hydrophobic environment represented by adding 50% trifluoroethanol to the above buffer (FIG. 4 and Table 10). Peptides D26 (SEQ ID NO:3) and the 11 analogs (D51-D61; SEQ ID NO.4-8, 11-13, 15-17) all have specificity determinants (positively charged residues at positions 13 and 16 on the non-polar face) which dramatically reduces the α-helical structure in aqueous conditions at pH 7 (FIG. 4, Table 10).

The α-helical content of D26 was 8.9% and the 11 analogs varied from a low of 3.3% to a high of 13.7% (Table 10). In the hydrophobic medium containing 50% TFE, helical structure was induced in all 11 analogs and varied from a low of 27.2% (D56) to a high of 100% (D52). The inducible helical structure is shown by A[O]222 nm TFE-Aqueous which varied from 392(D56) to 1422(D52) (Table 10). All peptides with specificity determinants show the desired properties of low α-helical structure in aqueous conditions and inducible α-helical structure in the presence of a hydrophobic environment which suggests that the peptides will take up an α-helical structure in the hydrophobicity of the bacterial membrane. In peptides D51-D55 the position of positively charged residues on the polar face is varied with D52 having the most inducible α-helical structure when in a hydrophobic environment (Table 10). In peptide D52 the positively charged residues on the polar face are located in two groups K3 and K4 at the N-terminal end of the peptide and K22, K25 and K26 at the C-terminal end (FIGS. 1 and 2).

In the case of D52 analogs the specificity determinants are varied from K13/K16 in D52, Orn 13/Orn 16 in D56, Dbu 13/Dbu 16 in D57 and Arg 13/Arg 16 in D58 (Table 1B from patent). These changes in the type of positively charged residue on the non-polar face as specificity determinants does affect the α-helical structure in aqueous conditions and the inducible helical structure in the presence of 50% TFE (Table 10). When Lys is replaced by Orn, Dbu or Arg helical structure (compared to D52) is dramatically reduced in aqueous conditions (from 13.7% to 3.5%, 5.9% and 7.2% for D56, D57 and D58) and inducible structure is also reduced from A[O]222 of 1422 for D52 to 392, 574 and 750 for D56, D57 and D58. These results show that the type of positively charged residue used for specificity determinants affects the helical properties of the peptides (Table 10).

The type of positively charged residue used on the polar face was also varied. D52 contains 5 Lys residues on the polar face whereas D59, D60 and D61 contain 5 Orn, 5 Dbu and 5 Arg residues at the same positions (Table 1C in patent application). These substitutions also affect the % helix in aqueous conditions D52 (13.7%), D59 (6.9%), D60 (3.3%) and D61 (10.6%). Inducible α-helical structure is also affected Δ[Θ]222 varying from 1422 for D52 to 1061 for D59, 881 for D60 and 835 for D61. Clearly these results show that the type of positively charged residue used on the polar face affects the helical properties of the peptides (Table 10).

### Example 4: Peptide Self-Association

Peptide self-association, the ability to oligomerize/dimerize in aqueous solution, is an important parameter that can improve antimicrobial activity while removing toxicity. The inventors hypothesize that the monomeric random-coil antimicrobial peptides in aqueous solution are best suited to pass through a polysaccharide capsule, the outer membrane lipopolysaccharide and the cell wall peptidoglycan layer of microorganisms prior to penetration into the cytoplasmic membrane, induction of alpha-helical structure and disruption of membrane structure to kill target cells. On the other hand, if the self-association ability of an AMP in aqueous medium is too strong, stable folded oligomers/dimers through interaction of their non-polar faces are formed which decreases the ability of the AMP to dissociate to monomer and the dimer/oligomer to effectively pass through the capsule and cell wall to reach the cytoplasmic membrane. In the present study, the ability of the AMPs to self-associate was determined by a technique developed in the inventors' laboratory, referred to as RP-HPLC temperature profiling at pH 2 over the temperature range of 5° C. to 80° C. This novel method to measure self-association of small cyclic beta-sheet AMPs was first reported by Lee and co-workers in 2003 and is a key method in the design and optimization of amphipathic alpha-helical AMPs.

Temperature Profiling of Peptides on Reversed-Phase HPLC:

Purified peptides were analyzed on an Agilent 1200 series liquid chromatograph for temperature profiling using a Zorbax 300 SB-C8 column (150 mm×2.1 mm I.D.; 5 μm particle size, 300 Å pore size) from Agilent Technologies. Conditions: linear AB gradient (0.25% acetonitrile/min) and a flow rate of 0.30 ml/min, where eluent A was 0.20% aqueous TFA, pH 2 and eluent B was 0.18% TFA in acetonitrile.

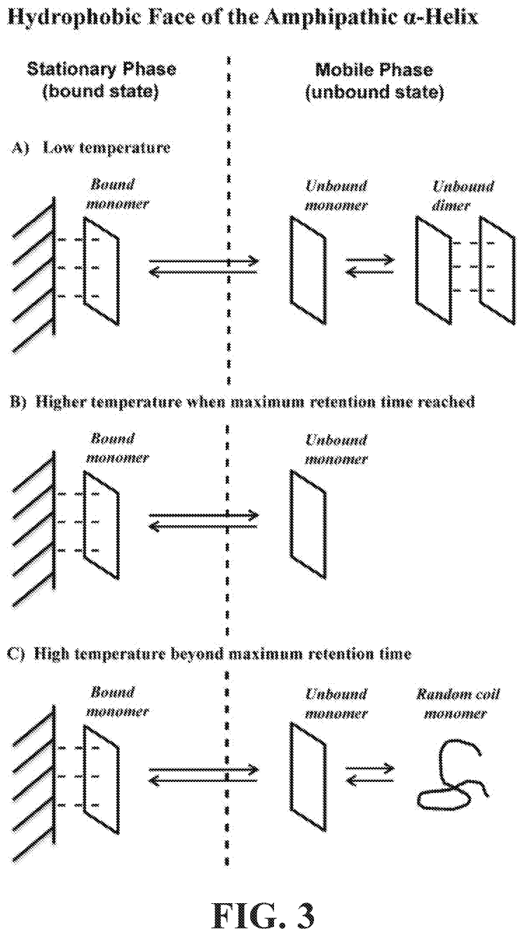

It is important to understand how the RP-HPLC temperature profiling method works. At low temperature, AMPs are capable of self-associating in aqueous solution via their non-polar faces. As shown in FIG. 3, equilibrium is established between monomer and dimer and the concentration of monomer and dimer at any given temperature depends on the strength of the hydrophobic interactions between the two monomers to form the alpha-helical folded dimer. In RP-HPLC, the hydrophobicity of the matrix disrupts or dissociates the dimer and only the monomeric form of the peptide is bound to the hydrophobic matrix by its preferred binding domain (non-polar face). The monomeric form of the peptide can partition between the hydrophobic surface of the alkyl ligands on the reversed-phase matrix and the mobile phase. At low temperature, the monomer can dimerize in the mobile phase and the retention time is decreased due to the large population of dimers in solution. At higher temperatures, the population of dimers in the mobile phase during partitioning decreases, which increases the concentration of monomeric peptide in solution and thereby increases retention time. At some higher temperature, no dimer exists in the mobile phase and the peptide has its maximum retention time. At temperatures beyond the point of maximum retention time the unbound helical peptide in the mobile phase is in equilibrium with the random-coil conformation of the peptide and retention time decreases with further increasing temperature. With the random coil control peptide that does not dimerize, the peptide binds to the stationary phase and partitions in the mobile phase as a monomer with undefined structure throughout the temperature range (5°-77° C.) (FIG. 3).

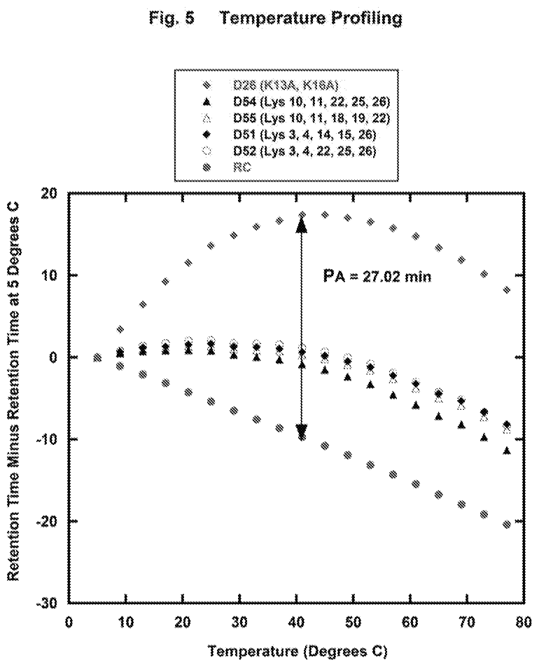

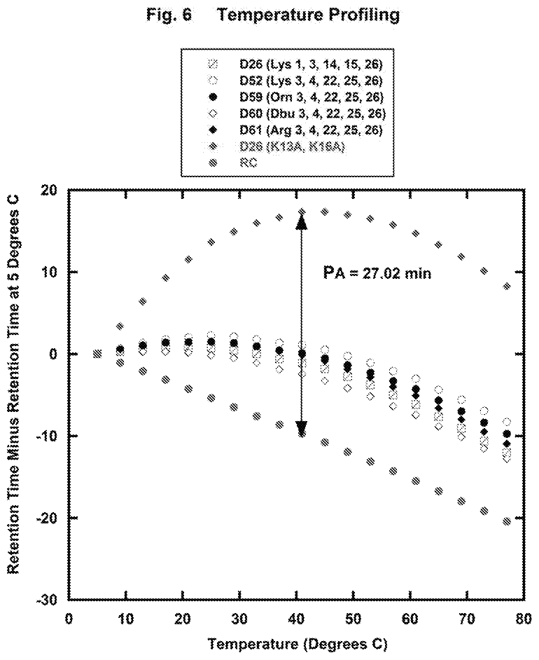

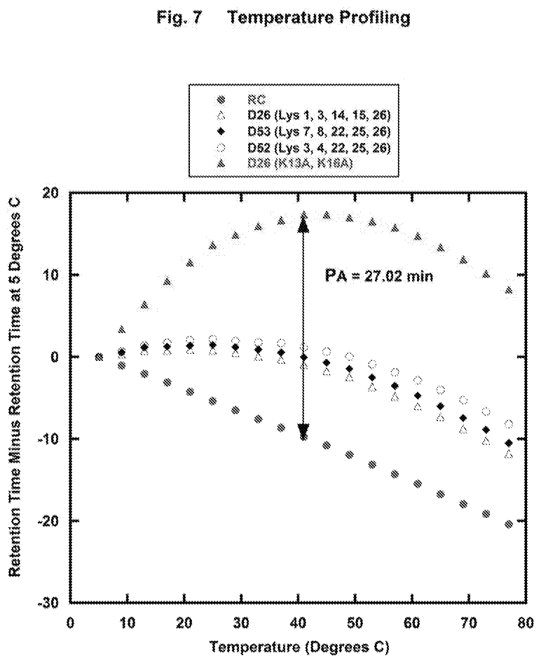

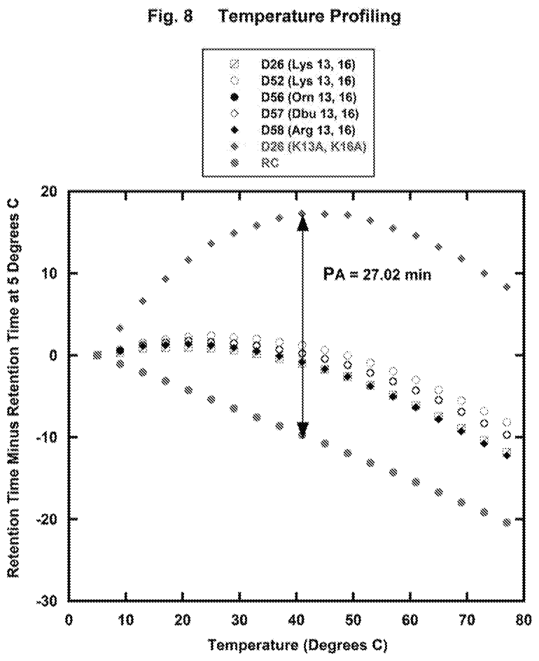

FIGS. 5-8 illustrate self-association profiles of the α-helical AMP analogs as determined by temperature profiling in RP-HPLC. Thus, retention behavior of the peptides from RP-HPLC over a temperature range of 5° C.-77° C. was normalized to their retention times at 5° C. At 5° C., the low temperature, combined with the α-helix inducing nature of the hydrophobic stationary phase (analogous to the helix inducing properties of 50% aq. TFE; see FIG. 4 and Table 10) will maximize the helical potential of the peptides. As noted above (FIG. 3), the concentration of monomer and dimer (the latter being formed by peptide self-association due to interactions between the non-polar faces) at any given temperature depends on the strength of the hydrophobic interactions between the two monomers. The magnitude of peptide self-association is then quantified by the relative rise in peptide retention time with increasing temperature relative to the retention time at 5° C. and, subsequently, in its relation to the retention time profile of the random coil peptide standards. Thus, the peptide self-association parameter, P_{A}, represents the maximum change in peptide retention time relative to the random coil peptide standards (RC in FIGS. 5-8; Table 11). Thus, the higher the P_{A} value, the greater the self-association.

From FIGS. 5-8, the peptide analog with no specificity determinants (no positively charged Lys residues at positions 13 and 16) in the center of the non-polar face of the amphipathic α-helical peptide unsurprisingly exhibits the highest P_{A} value of 27.02 min at a Tp (temperature at which maximum retention is observed over the temperature range 5° C.-77° C.; Table 11) of 41° C. That is, this peptide is exhibiting considerable self-association. In contrast, the remaining analogs, all of which contain the same number of positively charged residues (Lys, Orn, Dbu or Arg) in the polar face or non-polar face, exhibit considerably lower P_{A} values, ranging from just 3.41 min to 7.78 min at Tp values of only 17° C.-25° C. Thus, the presence of the positively charged residues on the polar or non-polar faces of the peptides is resulting in essentially much reduced peptide self-association, a desired property of our AMPs.

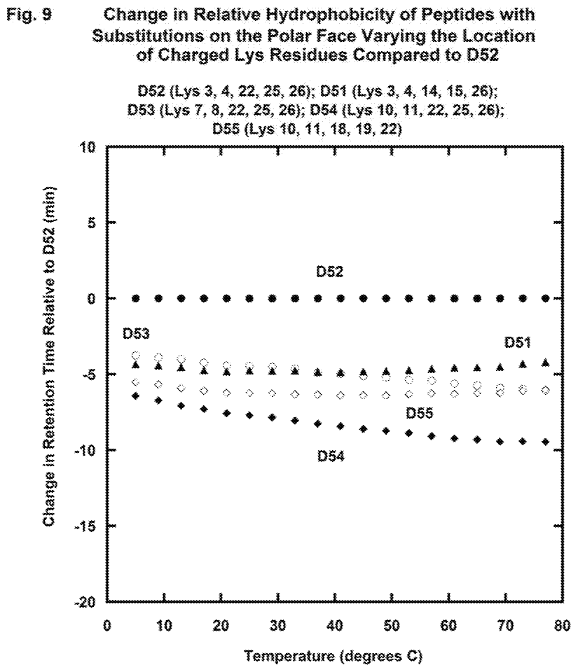

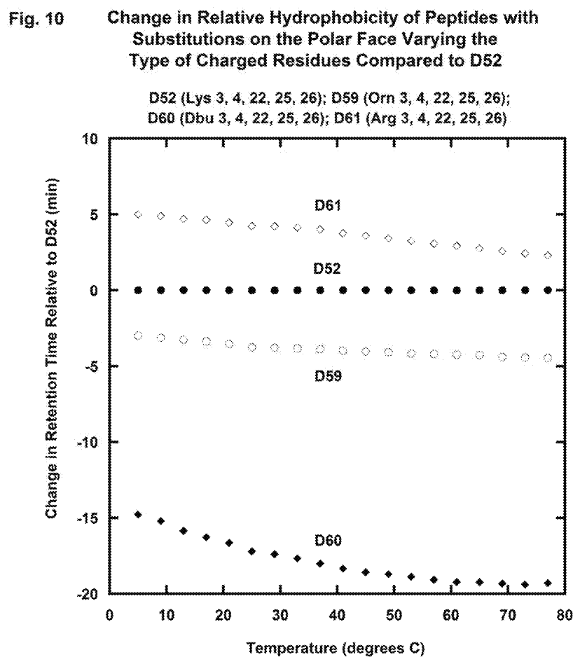

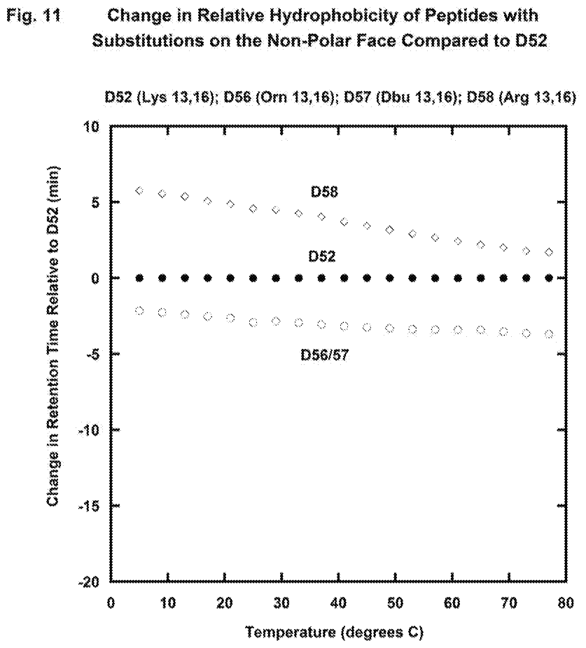

FIGS. 9-11 compare the change in relative hydrophobicity of the peptides depending on the location (FIG. 9) or type of positively charged residues (Lys, Orn, Dbu or Arg) on the polar face (FIG. 10) or non-polar face (FIG. 11) of the peptides. From FIGS. 9-11, even though the P_{A} values of these analogs is low compared to the D26 (K13A, K16A) standard (Table 11), i.e., self-association is of a low magnitude, there does appear to be an approximate correlation with P_{A} values relative to that of D52 and the variation of apparent peptide hydrophobicity relative to D52 with increasing temperature. Thus, the lower the P_{A} value of a peptide compared to that of D52, the more the apparent hydrophobicity deviates from that of D52. For example, D60 (P_{A}=3.41, the lowest value of the analogs examined) exhibits the greatest deviation from D52 (P_{A}=7.78); in contrast, peptides with P_{A} values closer to that of D52 generally do not deviate as much. These results could reflect that the lesser self-association of analogs compared to D52 leads to them more quickly exposing their overall total hydrophobicity as expressed by RP-HPLC retention time, with an increase in temperature, i.e., the monomer-dimer equilibrium in solution is being more quickly disrupted by an increase in temperature relative to D52. It is interesting, that D60 with the lowest P_{A} value (3.41) and the lowest TP value (17° C., Table 11) has the highest HC₅₀ value (that is, D60 is not hemolytic) and has the highest therapeutic index of the eleven analogs (D51-D61) (Table 12).

### Example 5: Antibacterial Activity

Gram-Negative Bacterial Strains used in this Study: All the A. baumannii strains used in this study were 1) obtained from the collection of Dr. Anthony A. Campagnari at the University of Buffalo and originally isolated from different patients and organs/tissues (strain 649, blood; strain 689, groin; strain 759, gluteus; strain 884, axilla; strain 985, pleural fluid); 2) were purchased from the American Type Culture Collection (ATCC, Manassas, Va., USA) (strain ATCC 17978, fatal meningitis; and strain ATCC 19606, urine); 3) obtained from MERCK (M89941, M89949, M89951, M89952, M89953, M89955 and M89963). These seven A. baumannii strains were resistant to polymyxin B and polymyxin E (Colistin).

Gram Positive Bacterial Strains Used in this Study:

All the S. aureus strains used in this study were 1) nine methicillin-sensitive S. aureus strains; M22315, M22274 (Spine), M22300 (Finger), M22287 (Hip), M22312 (Finger), M22075 (Axilla), M21913 (Finger), BL7429 (Blood) and M22097 (Neck) 2) eight Methicillin/Oxacillin-resistant S. aureus strains; M22424 (arm), M22111 (ear), M22360 (labia), M22354, M21756 (nose), M22130, M22224 (leg), M21742 (nose).

Measurement of Antimicrobial Activity (MIC):

The minimal inhibitory concentration (MIC) is defined as the lowest peptide concentration that inhibited bacterial growth. MICs were measured by a standard microtiter dilution method in Mueller Hinton (MH) medium. Briefly, cells were grown overnight at 37° C. in MH broth and were diluted in the same medium. Serial dilutions of the peptides were added to the microtiter plates in a volume of 50 microL, followed by the addition of 50 microL of bacteria to give a final inoculum of 5×10⁵ colony-forming units (CFU)/mL. The plates were incubated at 37° C. for 24 h, and the MICs were determined. Table 12 shows the antimicrobial activity of D26 and eleven analogs (D51-D61) against seven strains of Acinetobacter baumannii that are resistant to polymyxin B and colistin (antibiotics of last resort) (Table 12). The antimicrobial activity does not deviate significantly, with the geometric mean MIC value varying from a 0.4 to 1.0 micromolar (Table 12).

### Example 6: Hemolytic Activity and Therapeutic Indices

Measurement of Hemolytic Activity:

Peptide samples (concentrations determined by amino acid analysis) were added to 1% human erythrocytes in phosphate-buffered saline (100 mM NaCl, 80 mM Na₂HPO₄, 20 mM NaH₂PO₄, pH 7.4) and the reaction mixtures were incubated at 37° C. for 18 h in microtiter plates. Two-fold serial dilutions of the peptide samples were carried out. This determination was made by withdrawing aliquots from the hemolysis assays and removing unlysed erythrocytes by centrifugation (800×g). Hemoglobin release was determined spectrophotometrically at 570 nm. The control for 100% hemolysis was a sample of erythrocytes treated with water. The control for no release of hemoglobin was a sample of 1% erythrocytes without any peptide added. Since erythrocytes were in an isotonic medium, no detectable release (<1% of that released upon complete hemolysis) of hemoglobin was observed from this control during the assay. The hemolytic activity is generally determined as the peptide concentration that causes 50% hemolysis of erythrocytes after 18 h (HC₅₀). HC₅₀ was determined from a plot of percent lysis versus peptide concentration (microM). If 50% hemolysis could not be reached, the inventors used HC₃₀ values (30% hemolysis).

Calculation of Therapeutic Index

The therapeutic index is a widely-accepted parameter to represent the specificity of antimicrobial peptides for prokaryotic versus eukaryotic cells. It is calculated by the ratio of hemolytic activity and antimicrobial activity (MIC); thus, larger values of therapeutic index indicate greater specificity for prokaryotic cells. With the peptides used in this study the inventors used the HC₃₀/MIC ratio value to calculate the therapeutic index. The hemolytic activity and therapeutic index for D26 and the eleven analogs (D51-D61) are shown in Table 12. The hemolytic activity ranges from HC₅₀ values of 3.4 μM for D61 to high values of >1776 (D26) and >1880 (D60). The therapeutic indices, which is the HC₅₀ value divided by the MIC_{GM} values, range from 6.8 to >1880 (Table 12). It is interesting that the T.I. value of 6.8 for Arg in D61 is contrasted with Dbu residues in D60 at >1880. This indicates that the subtle change in the type of positively charged residue used in the AMP can have a dramatic effect on the biological activity of the desired AMP.

### Example 7: Gram-Negative Pathogen Selectivity

The inventors have shown that the substitution of one or two specificity determinant(s) in broad spectrum native AMPs, Piscidin 1 and Dermaseptin S4 resulted in new AMPs that encode selectivity for Gram-negative pathogens and remove both Gram-positive activity and hemolytic activity from broad-spectrum AMPs. The Gram-negative selectivity factor for D-Piscidin 1 (19K) (one-specificity determinant) resulted in a 55-fold improvement in selectivity (MIC_{GM}, S. aureus/MIC_{GM} A. baumannii) and D-Dermaseptin S4 (L7K, A14K) (two-specificity determinants) resulted in a >99-fold improvement in A. baumannii selectivity compared to S. aureus. These results suggested that amphipathic alpha-helical AMPs can be designed with selectivity for Gram-negative pathogens.

D26 is a unique AMP in that it is also more selective for the Gram-negative pathogen A. baumannii than for the Gram-negative pathogen P. aeruginosa. There is a 25-fold improvement in selectivity for (MIC_{GM}, P. aeruginosa/MIC_{GM}, A. baumannii). By comparison, D16 has a selectivity factor of 2.7 (Table 8).

### Example 8: Antimicrobial Activity of AMPs in the Presence of Human Sera

A critical component to the systemic use of AMPs to treat bacterial infections is the extent of AMP binding to serum proteins. In addition, since only the unbound AMP is available to interact with the therapeutic target, the extent of serum binding can have significant effects on efficacy. To address this issue, the inventors determined the MIC values of these peptide candidates in the presence of Mueller Hinton (MH) medium and MH medium supplemented with human sera (25% v/v). This assay estimates the in vivo bioavailability of the AMPs. The appropriate non-specific affinity of a drug for serum proteins can significantly improve in vivo half-life and decrease clearance. An increase in MIC in serum is attributed to inhibition of antimicrobial activity due to serum protein binding.

What is claimed is:

1. An antimicrobial peptide (AMP) comprising the amino acid sequence:
(SEQ ID NO: 1)
X¹-L-X²-X³-L-L-X⁴-X⁵-L-X⁶-X⁷-A-X⁸-X⁹-X¹⁰-X¹¹-L-X¹²-X¹³-
L-L-X¹⁴-A-L-X¹⁵-X¹⁶
wherein:
each residue is in the D-enantiomeric form;
X¹ is an amino acid in the D-enantiomeric form selected from Lysine (K; Lys), Arginine (R; Arg), Ornithine (O; Orn), and Alanine (A; Ala);
each of X², X³, X⁴, X⁶, X⁷, X¹², X¹⁴, and X¹⁵ are independently, amino acids in the D-enantiomeric form selected from Lysine (K; Lys), Arginine (R; Arg), Ornithine (O; Orn), Diaminobutyric acid (Dbu), and Serine (S; Ser);
each of X⁵, X¹³, X¹⁶ are independently, amino acids in the D-enantiomeric form selected from Lysine (K; Lys), Arginine (R; Arg), Ornithine (O; Orn), Diaminobutyric acid (Dbu), and Serine (S; Ser), and Threonine (T; Thr);
each of X⁸, X¹¹ are independently, amino acids in the D-enantiomeric form selected from Lysine (K; Lys), Serine (S; Ser), and Threonine (T; Thr), Arginine (R; Arg), Ornithine (O; Orn), Alanine (A; Ala), Diaminobutyric acid (Dbu), and Diaminopropionic acid (Dpr); and,
each of X⁹ and X¹⁰ are independently, amino acids in the D-enantiomeric form selected from Lysine (K; Lys), Arginine (R; Arg), Ornithine (O; Orn), Serine (S; Ser), and Alanine (A; Ala);
wherein the AMP comprises two positively charged residues on the non-polar face and has 5 positively charged residues on the polar face, has a total charge of +7.

2. An AMP of claim 1, wherein the AMP has amino acid sequence (referring to the single-letter amino acid code), entirely in the D-enantiomeric form:
(SEQ ID NO: 10)
ALKKLLSTLSSA X⁸ SS X¹¹ LSTLLKALKK
wherein each of X⁸, X¹¹ are independently amino acids in the D-enantiomeric form selected from Lysine (K; Lys), Arginine (R; Arg), Ornithine (O; Orn), and Diaminobutyric acid (Dbu).

3. An AMP of claim 1, wherein the AMP has amino acid sequence (referring to the single-letter amino acid code), entirely in the D-enantiomeric form:
(SEQ ID NO: 14)
AL X² X³ LLSTLSSAKSSKLSTLLX¹⁴ AL X¹⁵ X¹⁶
wherein each of X², X³, X¹⁴, X¹⁵, X¹⁶ are independently amino acids in the D-enantiomeric form selected from Lysine (K; Lys), Arginine (R; Arg), Ornithine (O; Orn), and Diaminobutyric acid (Dbu).

4. The AMP of claim 1, wherein the AMP comprises an amino acid sequence selected from the group consisting of:

| Sequence | SEQ ID NO |
|---|---|
| KLKSLLSTLSSAKKKKLSTLLSALSK | 3 |
| ALKKLLSTLSSAKKKKLSTLLSALSK | 4 |
| ALKKLLSTLSSAKSSKLSTLLKALKK | 5 |
| ALSSLLKKLSSAKSSKLSTLLKALKK | 6 |
| ALSSLLSTLKKAKSSKLSTLLKALKK | 7 |
| ALSSLLSTLKKAKSSKLKKLLKALSS | 8 |
| ALKKLLSTLSSAOrnSSOrnLSTLLKALKK | 11 |
| ALKKLLSTLSSADbuSSDbuLSTLLKALKK | 12 |
| ALKKLLSTLSSAArgSSArgLSTLLKALKK | 13 |
| ALOrnOrnLLSTLSSAKSSKLSTLLOrnALOrnOrn | 15 |
| ALDbuDbuLLSTLSSAKSSKLSTLLDbuALDbuDbu | 16 |
| ALArgArgLLSTLSSAKSSKLSTLLArgALArgArg | 17 |

5. The AMP of claim 1, wherein the amino acid sequence of the AMP comprises a sequence selected from the group consisting of SEQ ID NOs: 1, 3-8 and 10-17

6. The AMP of claim 1, wherein the amino acid sequence of the AMP comprises the sequence of SEQ ID NO:3.

7. The AMP of any one of claims 1-6, wherein the AMP inhibits propagation of a prokaryote.

8. The AMP of claim 7, wherein the prokaryote is a Gram-negative bacterium.

9. The AMP of claim 8, wherein the Gram-negative bacterium is at least one of A. baumannii and P. aeruginosa.

10. The AMPs of any one of claims 1-6, wherein the therapeutic index (calculated by the ratio of hemolytic activity and antimicrobial activity (MIC)) is at least 70.

11. The AMPs of any one of claims 1-6, wherein the therapeutic index (calculated by the ratio of hemolytic activity and antimicrobial activity (MIC)) is between 70 and 1600.

12. The AMP of any one of claims 1-6, wherein the AMP exhibits greater antimicrobial activity against Gram-negative P. aeruginosa or Acinetobacter baumannii drug-resistant mutants compared to other AMPs.

13. The AMP of any one of claims 1-6, wherein the AMP exhibits at least a 3-fold greater antimicrobial activity against Gram-negative P. aeruginosa or Acinetobacter baumannii compared to other AMPs.

14. The AMP of any one of claims 1-6, wherein the AMP exhibits at least a 10-fold increased selectivity for prokaryotic cells over eukaryotic cells.

15. The of AMP of claim 14, wherein the Gram-negative bacteria is Acinetobacter baumannii, and the Gram-positive bacteria is Staphylococcus aureus.

16. The AMP of any one of claims 1-6, wherein the AMP is equally effective in inhibiting the propagation of an antibiotic-resistant prokaryote and an antibiotic-sensitive prokaryote.

17. A pharmaceutical composition comprising at least one peptide of any one of claims 1-6, and a pharmaceutically acceptable carrier.

18. The pharmaceutical composition of claim 17, comprising a mono-phasic pharmaceutical composition suitable for parenteral or oral administration consisting essentially of a therapeutically-effective amount of at least one peptide of claim 1, and a pharmaceutically acceptable carrier.

19. A method of preventing or treating a microbial infection comprising administering to a subject in need thereof a therapeutically effective amount of at least one peptide of any one of claims 1-6, or a pharmaceutical composition of claim 17.

20. The method of claim 19, wherein the microbial infection is a bacterial infection.

21. The method of claim 20, wherein the bacterial infection is a Gram-negative bacterial infection.

22. The method of claim 20, wherein the bacterial infection is an antibiotic resistant bacterial infection.

23. The method of claim 20, wherein an infecting microorganism is at least one of Acinetobacter baumannii and Pseudomonas aeruginosa.

24. The method of claim 20, wherein an infecting microorganism is multi-drug resistant Pseudomonas aeruginosa or Acinetobacter baumannii.

25. The method of claim 19, wherein the administration of the peptide or pharmaceutical composition is by an administration route selected from oral, topical, intravenous, intraperitoneal, intramuscular, intradermal, intrasternal, intraarticular injection, intrathecal, and infusion.

26. The method of claim 19, wherein the peptide or pharmaceutical composition is administered in conjunction with one or more additional antimicrobial agents.

27. A method of preventing a microbial infection in an individual at risk of developing an infection comprising administering an effective amount of at least one peptide of any one of claims 1-6, or a pharmaceutical composition of claim 17, to the individual in need thereof.

28. The method of claim 27, wherein the individual is a surgical patient.

29. The method of claim 27, wherein the individual is a hospitalized patient.

30. A method of combating a bacterial infection in a patient, comprising applying at least one peptide of any one of claims 1-6 or a pharmaceutical composition of claim 17 to a body surface of the patient.

31. The method of claim 28, wherein the body surface is a wound.

32. The method of claim 28, wherein the composition is applied following an operation or surgery.

33. At least one peptide of any one of claims 1-6, or a pharmaceutical composition of claim 17 for use in the treatment of a microbial infection.

34. Use of at least one peptide of any one of claims 1-6, or a pharmaceutical composition of claim 17 in the manufacture of a medicament for the prevention or treatment of a microbial infection.

35. The AMP of any one of claims 1-6, wherein the AMP has one or more improved biological properties selected from improved antimicrobial activity against a Gram-negative microorganism, decreased hemolysis of human red blood cells, decreased binding to human serum proteins, and improved therapeutic index for a Gram-negative microorganism.

36. A pharmaceutical composition of claim 17 in combination with one or more further therapeutic agents.

37. A pharmaceutical composition of claim 36 wherein the one or more further therapeutic agents are each independently selected from antibiotics.

38. A peptide of any one of claims 1-6 for use as a pharmaceutical.
